(19) United States
(12) Patent Application Publication
(10) Pub. No.: US 2022/0175989 A1
(43) Pub. Date: Jun. 9, 2022

(54) DISINFECTION DEVICE

(71) Applicants: AXIS LIGHTING INC., Lasalle (CA); STERILUMEN, INC., Tarrytown, NY (US)

(72) Inventors: Howard YAPHE, Lasalle (CA); Andrew MILES, Lasalle (CA); Nathanel TITANE, Lasalle (CA); Hristea MIHALCEA, Lasalle (CA); Donald SIMMONS, Tarrytown, NY (US)

(21) Appl. No.: 17/543,425

(22) Filed: Dec. 6, 2021

Related U.S. Application Data
(63) Continuation-in-part of application No. 17/114,447, filed on Dec. 7, 2020.

(51) Int. Cl. A61L 2/10; A61L 2/26

(52) U.S. Cl. CPC A61L 2/10; A61L 2202/25; A61L 2/26

(57) ABSTRACT

Disclosed is a disinfection device for controllably emitting and directing disinfecting UV energy to at least one elongate first target location and at least one elongate second target location in a user space. The disinfection device comprises a housing structure mountable on a support structure and having an outer boundary. The outer boundary is configured to provide a first array of outputs to emit and direct disinfecting UV energy to the at least one first target location, a second array of outputs to deliver directed disinfecting UV energy to the at least one second target location.

### REFERENCE TO CO-PENDING APPLICATION

This application is a Continuation-in-Part of co-pending U.S. patent application Ser. No. 17/114,447, filed Dec. 7, 2020, entitled DISINFECTION DEVICE, the disclosure of which is hereby incorporated by reference in its entirety.

### FIELD OF THE DISCLOSURE

The present disclosure relates to disinfection devices and disinfection systems using disinfection devices and associated structures, for use in interior or exterior user locations in a user space for directed emissions of UV energy to disinfect contact surfaces therein.

### BACKGROUND

The COVID-19 pandemic that started in 2020 has reinforced the need for improved control of pathogens in locations of user interaction that have user contact surfaces (or tough points), that is specific locations where people make contact with the same location, such as a tap in a bathroom, or a button on a parking meter or kiosk, as examples. Usually, this involves a manual cleaning to sterilize these kinds of surfaces. However, the degree of disinfection relies, in all cases, on the care and attention of the person performing the task.

There remains a need to improve modes of disinfecting surfaces, by way of devices that may be integrated into fixtures adjacent to, or providing, user contact surfaces.

### SUMMARY

An aspect may provide a disinfection device for controllably emitting and directing disinfecting UV energy to at least one first linear target location and at least one second linear target location in a user space, comprising a linear housing structure mountable on a support structure and having an outer boundary. The outer boundary may be configured to provide:
a first linear array of outputs to deliver directed disinfecting UV energy to the at least one first linear target location, with each output of the first array thereof configured to receive directed disinfecting UV energy from one instance of a first array of disinfecting UV energy directors, with each disinfecting director of the first array thereof configured to receive disinfecting UV energy from one instance of a first array of disinfecting UV energy emitters; and
a second linear array of outputs to deliver directed disinfecting UV energy to the at least one second linear target location, with each output of the second array thereof configured to receive directed disinfecting UV energy from one instance of a second array of disinfecting UV energy directors, with each disinfecting director of the second array thereof configured to receive disinfecting UV energy from one instance of a second array of disinfecting UV energy emitters.

Another aspect may provide a disinfection device for controllably emitting and directing disinfecting UV energy to at least one first elongate target location and at least one second elongate target location in a user space. The device may comprise a housing structure mountable on a support structure and having an outer boundary, wherein the outer boundary may be configured to provide:
a first array of outputs to deliver directed disinfecting UV energy to the at least one first elongate target location, with each output of the first array thereof configured to receive directed disinfecting UV energy from one instance of a first array of disinfecting UV energy directors, with each disinfecting director of the first array thereof configured to receive disinfecting UV energy from one instance of a first array of disinfecting UV energy emitters; and
a second array of outputs to deliver directed disinfecting UV energy to the at least one second elongate target location, with each output of the second array thereof configured to receive directed disinfecting UV energy from one instance of a second array of disinfecting UV energy directors, with each disinfecting director of the second array thereof configured to receive disinfecting UV energy from one instance of a second array of disinfecting UV energy emitters.

Another aspect may provide a disinfection device for controllably emitting and directing disinfecting UV energy to at least one first target location and at least one second target location in a user space. The device may comprise a linear housing structure mountable on or in at least one support structure and having an outer boundary, wherein the outer boundary is configured to provide:
at least one output configured:
   to receive a first directed disinfecting UV energy from at least one instance of a first array of disinfecting UV energy directors, with each UV energy disinfecting director of the first array thereof configured to receive a first disinfecting UV energy from one instance of a first array of disinfecting UV energy emitters, and to deliver the first directed disinfecting UV energy to the at least one first target location; and
   to receive a second directed disinfecting UV energy from at least one instance of a second array of disinfecting UV energy directors, with each disinfecting UV energy director of the second array thereof configured to receive a second disinfecting UV energy from one instance of a second array of disinfecting UV energy emitters, and to deliver the second directed disinfecting UV energy to the at least one second target location.

Another aspect may provide a disinfection device for controllably emitting and directing disinfecting UV energy to at least one first proximal target location defined in a first plane, and at least one second distal target location defined in a second plane, in a user space. The device may comprise a housing structure mountable on or in at least one support structure and having an outer boundary, wherein the outer boundary is configured to provide:
at least one output configured:
   to receive a first directed disinfecting UV energy from a first array of at least one first disinfecting UV engine to deliver the first directed disinfecting UV energy to the at least one first proximal target location; and
   to receive a second directed disinfecting UV energy from a second array of second disinfecting UV engines to deliver the second directed disinfecting UV energy to the at least one second distal target location.

Another aspect may provide a disinfection device for controllably emitting and directing disinfecting UV energy to at least one elongate first target location and at least one second target location in a user space, comprising a housing structure mountable on a support structure and having an outer boundary, wherein the outer boundary is configured to provide:
a first array of outputs to deliver directed disinfecting UV energy to the at least one first target location, with each output of the first array thereof configured to receive directed disinfecting UV energy from one instance of a first array of disinfecting UV energy directors, with each disinfecting director of the first array thereof configured to receive disinfecting UV energy from one instance of a first array of disinfecting UV energy emitters; and
a second array of outputs to deliver directed disinfecting UV energy to the at least one second target location, with each output of the second array thereof configured to receive directed disinfecting UV energy from one instance of a second array of disinfecting UV energy directors, with each disinfecting director of the second array thereof configured to receive disinfecting UV energy from one instance of a second array of disinfecting UV energy emitters.

Each instance of the disinfecting UV energy directors of the first array thereof, and the corresponding instance of the disinfecting UV energy emitters of the first array thereof may be positioned adjacent a common location on a first mounting surface in the housing structure.

Each instance of the disinfecting UV energy directors of the second array thereof, and the corresponding instance of the disinfecting UV energy emitters of the second array thereof may be positioned adjacent a common location on a second mounting surface in the housing structure.

The outer boundary of the linear housing structure may be defined by an outer boundary wall, for example with each incidence of the first array of outputs and each incidence of the second array of outputs including a passage through the boundary wall.

The first and second arrays of outputs may extend along respective axes, with the axes being substantially parallel or substantially in common.

The outer boundary wall may have a continuous or discontinuous cross section.

The outer boundary wall may have a curved, linear and/or curvilinear cross section.

The boundary layer may have at least one ellipsoidal end region, with a pair of pair of opposed wall sections extending therefrom.

The opposed wall sections may be oriented with an acute included angle.

The support structure may include a base with an inclined support surface to engage one of the opposed wall sections.

The support structure may further comprise a first power supply segment, and the linear housing structure and support structure may each configured with a first set of contacts for coupling the first power supply segment to a second power supply segment in the linear housing structure to energize the first and second arrays of UV energy emitters.

The first and second arrays of UV energy emitters may include UV LEDs, and the second power supply segment may include at least one driver to drive the UV LEDs.

The at least one driver may be configured to exchange control data signals with a remote controller networked therewith.

The support structure may further comprise a first control data path segment, and the linear housing structure and support structure may be each configured with a second set of contacts for coupling the first control data path segment to a second control data path segment in the linear housing structure in communication with the at least one driver.

Another aspect may provide a disinfection device for controllably emitting and directing disinfecting UV energy to at least one first target location and at least one second target location in a user space, comprising a linear housing structure mountable on or in at least one support structure and having an outer boundary, wherein the outer boundary is configured to provide:
at least one output configured:
   to receive a first directed disinfecting UV energy from at least one instance of a first array of disinfecting UV energy directors, with each UV energy disinfecting director of the first array thereof configured to receive a first disinfecting UV energy from one instance of a first array of disinfecting UV energy emitters, and to deliver the first directed disinfecting UV energy to the at least one first target location; and
   to receive a second directed disinfecting UV energy from at least one instance of a second array of disinfecting UV energy directors, with each disinfecting UV energy director of the second array thereof configured to receive a second disinfecting UV energy from one instance of a second array of disinfecting UV energy emitters, and to deliver the second directed disinfecting UV energy to the at least one second target location.

Each instance of the disinfecting UV energy directors of the first array thereof, and the corresponding instance of the disinfecting UV energy emitters of the first array thereof may be positioned adjacent a common location on a first linear mounting surface in the linear housing structure.

Each instance of the disinfecting UV energy directors of the second array thereof, and the corresponding instance of the disinfecting UV energy emitters of the second array thereof may be positioned adjacent a common location on a second linear mounting surface in the linear housing structure.

The first and second linear surfaces may be defined by corresponding first and second linear LED board structures positioned in the linear housing structure.

The outer boundary of the linear housing structure may be defined by an outer boundary wall, with each incidence of the array of at least one output including a passage through the boundary wall.

The outer boundary wall may include at least one ellipsoidal end region, with a pair of pair of opposed wall sections extending therefrom.

The at least one passage may extend through the at least one ellipsoidal end region along a dimension parallel to a central axis thereof, and a dimension transverse to the central axis.

The at least one passage may be defined by a pair of opposed linear boundaries which are spaced by included radial angle therebetween.

The first and second linear LED board structures may each be configured to extend along a corresponding linear boundary, so as to cause to the first disinfecting UV energy of the first array to pass through the second directed disinfecting UV energy of the second array.

At least one UV energy transmissive cover structure may be provided to close the at least one passage.

The opposed linear boundaries may be oriented with an acute included angle relative to the central axis.

The support structure may include a base structure with a support surface to engage one of the opposed wall sections.

The support structure may include a base with a pair of opposed support surfaces, each to engage a corresponding one of the opposed wall sections, so as to position the linear housing structure into the support structure in a wedge-like configuration.

At least one anchor structure may be provided to anchor the linear housing structure in the support structure in the wedge-like configuration.

The linear housing structure and the support structure may further comprise complementary communication pathways for transferring power and/or communication signals therebetween.

The support structure and the linear housing structure may include respective adjacent surfaces in the wedge-like configuration with the communication pathways defined thereon.

Another aspect may provide a disinfection device for controllably emitting and directing disinfecting UV energy to at least one first proximal target location defined in a first plane, and at least one second distal target location defined in a second plane, in a user space, comprising a housing structure mountable on or in at least one support structure and having an outer boundary, wherein the outer boundary is configured to provide:
at least one output configured:
   to receive a first directed disinfecting UV energy from a first array of at least one first disinfecting UV engine to deliver the first directed disinfecting UV energy to the at least one first proximal target location; and
   to receive a second directed disinfecting UV energy from a second array of second disinfecting UV engines to deliver the second directed disinfecting UV energy to the at least one second proximal target location.

The first array may include at least one instance of the first disinfecting UV engines, and the second array includes at least two instances of the second disinfecting UV engines.

Instances of first disinfecting UV engines in the first array may be fewer in number than instances of second disinfecting UV engines in the second array, so as to accommodate for a relatively shorter distance for the first directed disinfecting UV energy to travel to the first proximal target location, and the relatively longer distance for the second directed disinfecting UV energy to travel to the second distal target location.

The first array of UV engines may be spaced at a first spacing, and the second array of UV engines are spaced at a second spacing, wherein the first spacing may be greater than the second spacing.

The second spacing may be about twice the first spacing.

The first array and second array may be aligned with spaced substantially parallel first and second axes.

The housing structure may include opposed outer panel structures configured to engage corresponding opposed inner panels structures in a wedge-like heat transfer configuration.

At least one anchor structure may be provided to anchor the linear housing structure in the support structure so as to establish a heat transfer interface between each outer panel structure and a corresponding inner panel structure.

The outer panel structures may be in a wedge-like configuration with the opposed inner panel structures.

The housing structure and the support structure may further comprise complementary communication pathways for transferring power and/or communication signals therebetween.

The housing structure may comprise data storage structure to receive and store authentication data identifying one or more parameters of the linear housing structure.

The housing structure may comprise a monitor to update the one or more parameters.

The support structure may comprise an authenticator to communicate with a power controller, wherein the authenticator is configured to direct the power controller to deliver power to the first and second arrays when at least one parameter meets a first designated condition, or to interrupt power when at least one parameter meets a second designated condition.

One or more of the first and second disinfecting UV engines may include a UV energy director which receives UV energy from a UV energy emitter.

The UV energy director may include an optical coupling between a UV energy diffuser structure, a UV energy collimator structure, and the UV energy emitter.

One or more of the first target location, the second target location, the housing structure, the support structure, the first mounting surface, the second mounting surface, the first array, the second array, the support structure, the base structure, the opposed support surfaces, the opposed wall sections, the outer panel structures, the inner panel structures may be, or may be correspondingly, linear in configuration.

Another aspect may provide a disinfection device for controllably emitting and directing disinfecting UV energy to at least one first target region and at least one second target adjacent location in a user space, comprising a housing structure mountable on or in at least one support structure, and having an outer boundary, wherein the outer boundary is configured to provide:

at least one output configured:

to receive a first directed disinfecting UV energy from at least one instance of a first array of disinfecting UV energy directors, with each UV energy disinfecting director of the first array thereof configured to receive a first disinfecting UV energy from one instance of a first array of disinfecting UV energy emitters, and to deliver the first directed disinfecting UV energy to the at least one first target location; and

to receive a second directed disinfecting UV energy from at least one instance of a second array of disinfecting UV energy directors, with each disinfecting UV energy director of the second array thereof configured to receive a second disinfecting UV energy from one instance of a second array of disinfecting UV energy emitters, and to deliver the second directed disinfecting UV energy to the at least one second target location.

Another aspect may provide a disinfection device for controllably emitting and directing disinfecting UV energy to at least one first target region and at least one second target region in an interior user space, comprising a housing structure containing a first array of at least one first disinfecting UV engine configured to deliver a first directed disinfecting UV energy to the at least one first target region, and a second array of at least one second disinfecting UV engine configured to deliver a second directed disinfecting UV energy to the at least one second target region, so as to form a disinfecting UV energy transition between the first and second regions, with each of the first and second disinfecting UV energies at a designated energy value to correspond to corresponding attribute of the first and second regions respectively.

Another aspect may provide a disinfection device for controllably emitting and/or directing disinfecting UV energy to a plurality of regions in an interior or exterior user space, comprising a first array of at least one first disinfecting UV engine configured to deliver a first directed disinfecting UV energy, and a second array of at least one second disinfecting UV engine configured to deliver a second directed disinfecting UV energy, wherein a first group of one or more of the first and/or second disinfecting UV engines is configured to deliver the first and/or second disinfecting energies to at least one first target region, and a second group of one or more of the first and/or second disinfecting UV engines is configured to deliver the first and/or second disinfecting energies to at least one second target region.

At least one housing structure may be provided to contain the first and second arrays.

The first and second groups may be configured to form a disinfecting UV energy transition between the first and second target regions, with each of the first and second disinfecting UV energies in the respective groups at a designated energy strength according to a corresponding attribute of the first and second regions respectively.

Each disinfecting UV engine of the first array may be configured to deliver the first directed disinfecting UV energy to a first field, and each disinfecting UV engine of the second array may configured to deliver the second directed disinfecting UV energy to a second field, wherein each of the first target region and second target regions may include at least one first field and/or at least one second field.

Each of the first and second fields may a proximal field and/or a distal field.

The first group may be oriented to deliver the first and/or second directed disinfecting UV energies to the first region, as a central inner sector on a target surface in the interior user space.

The second group may be oriented to deliver the first and/or second directed disinfecting UV energy to the second region, as a peripheral outer sector on the target surface in the user space.

The target surface may include and/or be defined on a sink, counter, and/or wall surface, at least in part.

The target surface may include and/or be defined on at least one surface of a wall, floor, door, light, article of furniture, curtain and/or a barrier, fixture, toilet, tub, shower, a wheeled or non-wheeled assistive device, ATM, parking kiosks, a user accessible key and/or access panel, at least in part.

Instances of the first disinfecting UV engine in the first array may be equal or unequal in number than instances of the second disinfecting UV engine in the second array, so as to enable collective amounts of the first and second directed disinfecting UV energies to be configured by the corresponding numbers of first and second disinfecting UV engines, at least in part.

The first array may include a plurality of first disinfecting UV engines which are spaced at a first spacing, and the second array may include a plurality of second disinfecting UV engines of the second array which may be spaced at a second spacing, wherein the first spacing may be greater than, equal to, or less than the second spacing, and/or wherein each of the disinfecting UV engines of the first or second arrays may be respectively interspersed between a pair of disinfecting UV engines of the second or first arrays.

The first spacing may be about twice the second spacing.

The first array and second array may be aligned with spaced substantially parallel first and second axes.

Another aspect may provide a disinfection device for controllably emitting and directing focused disinfecting UV energy to a plurality of regions in an interior or exterior user space, comprising one or more collections of disinfecting UV engines, configured in one or more groups of one or more disinfecting UV engines to deliver focused respective disinfecting UV energies to at least one first target region and at least one second target region, so as to define:
at least one transition between the first and second target regions, and/or
at least one transition between one of the first and second target regions and an adjacent peripheral region beyond the first and or second regions wherein the adjacent peripheral region receives substantially no disinfecting UV energy.

The transition may be defined by a change in disinfecting UV energy strength of a designated value, according to one or more predetermined, algorithmic, formulaic or probable ranges across the transition.

At least one of the first target region, the second target region, the first field, and/or the second field may be restricted, least in part.

Another aspect may provide an installation in a user space, comprising the disinfection device as defined in any of the preceding clauses, examples, example embodiments and/or claims herein.

Another aspect may provide a method for controllably directing focused disinfecting UV energy to a plurality of regions on a target surface in an interior or exterior user space, comprising:
designating, on the target surface, a plurality of regions;
providing one or more collections of disinfecting UV engines,
configuring the one or more collections in one or more groups of one or more disinfecting UV engines to deliver the corresponding focused disinfecting UV energies to at least one first target region and at least one second target region, so as to define:
at least one transition between the first and second target regions, and/or
at least one transition between one of the first and second target regions and an adjacent peripheral region beyond the first and or second regions wherein the adjacent peripheral region receives substantially no disinfecting UV energy.

The designating step may include establishing a grid of sites on the target surface so that at least a plurality of the sites are located in each of the first and second target regions, and measuring differences in the disinfecting UV energy according to predetermined, algorithmic, formulaic or probable ranges between them, to define the at least one transition.

Another aspect may provide a linear disinfection device for controllably emitting and directing disinfecting UV energy to at least one first proximal target location defined in a first plane, and at least one second distal target location defined in a second plane, in a user space, comprising a linear housing structure mountable on or in at least one support structure and having an outer boundary. The linear housing structure may contain a first array of first disinfecting UV engines configured to deliver a first directed disinfecting UV energy to the at least one first proximal target location, and/or a second array of second disinfecting UV engines configured to deliver a second directed disinfecting UV energy to the at least one second proximal target location, wherein the outer boundary may be configured to provide two or more common outputs extending along the linear housing structure. Each of the two or more common outputs may be configured to receive both the first directed disinfecting UV energy from the first array of first disinfecting UV engines and receive the second directed disinfecting UV energy from the second array of second disinfecting UV engines to deliver the first and second directed disinfecting UV energies to the first and second target locations.

Each of the first and second disinfecting UV engines may include a corresponding first and second UV energy director, each of which receives UV energy from a corresponding first and second UV energy emitter.

Each of the first and second UV energy directors may include an optical coupling between a corresponding first and second UV energy diffuser structure, a corresponding first and second UV energy collimator structure, and the corresponding first and second UV energy emitter.

One or more instances of the first disinfecting UV engines in the first array may fewer in number than instances of the second disinfecting UV engines in the second array, so as to enable collective amounts of the first and second directed disinfecting UV energy to be configured by the corresponding numbers of first and second disinfecting UV engines.

The first disinfecting UV engines of the first array may be spaced at a first spacing, and/or the second disinfecting UV engines of the second array may be spaced at a second spacing, and/or wherein the first spacing may be greater than the second spacing, and/or wherein each of the first disinfecting UV engines may interspersed between a pair of second disinfecting UV engines.

The first spacing may be about twice the second spacing.

The first array and second array may be aligned with spaced substantially parallel first and second axes.

The linear housing structure and the support structure may further comprise complementary communication pathways for transferring power and/or communication signals therebetween.

The linear housing structure may comprise data storage structure to receive and store authentication data identifying one or more parameters of the linear housing structure.

The linear housing structure may comprise a monitor to update the one or more parameters.

The support structure may comprise an authenticator to communicate with a power controller, wherein the authenticator may be configured to direct the power controller to deliver power to the first and second arrays when at least one parameter meets a first designated condition, and/or to interrupt power when at least one parameter meets a second designated condition.

The outer boundary may include an ellipsoidal end region extending along the linear housing structure and/or may be provided with the two or more common outputs in side-by-side relation therein, and/or with opposed outer panel structures extending therefrom.

The opposed outer panel structures may be oriented at an acute included angle therebetween.

The outer boundary of the linear housing structure may be defined by an outer boundary wall, and/or with the two or more common outputs defined by a corresponding two or more passages through the boundary wall.

The outer boundary wall may have at least one ellipsoidal end region, and/or with a pair of opposed outer panel structures extending therefrom.

Each of the two or more passages may extend through the at least one ellipsoidal end region along a dimension parallel to a central axis thereof, and/or a dimension transverse to the central axis.

Each of the two or more passages may defined by a pair of opposed linear boundaries which may be spaced by an acute included angle therebetween.

The first and second disinfecting UV engines may include corresponding first and second linear LED board structures, wherein each may be configured to extend along a corresponding linear boundary, which may be so as to cause to the first disinfecting UV energy of the first array to pass through the second directed disinfecting UV energy of the second array.

At least one UV energy transmissive cover structure may be provided to close each corresponding passage.

The opposed linear boundaries may be oriented with an acute included angle relative to the central axis.

Another aspect may provide a disinfection device for controllably emitting and/or directing disinfecting UV energy to a plurality of regions in an interior or exterior user space, comprising a first array of at least one first disinfecting UV engine means configured to deliver a first directed disinfecting UV energy, and a second array of at least one second disinfecting UV engine means configured to deliver a second directed disinfecting UV energy, wherein a first group of one or more of the first and/or second disinfecting UV engine means is configured to deliver the first and/or second disinfecting energies to at least one first target region, and a second group of one or more of the first and/or second disinfecting UV engine means is configured to deliver the first and/or second disinfecting energies to at least one second target region.

At least one housing means may be provided to contain the first and/or second arrays.

Each disinfecting UV engine means of the first array may be configured to deliver the first directed disinfecting UV energy to a first field, and/or each disinfecting UV engine means of the second array may be configured to deliver the second directed disinfecting UV energy to a second field, wherein each of the first target region and/or second target region may include at least one first field and/or at least one second field.

The first group may be oriented to deliver the first and/or second directed disinfecting UV energies to the first region, as a central inner sector on a target surface in the user space.

The second group may be oriented to deliver the first and/or second directed disinfecting UV energy to the second region, as a peripheral outer sector on the target surface in the user space.

Instances of the first disinfecting UV engine means in the first array may be equal or unequal in number than instances of the second disinfecting UV engine means in the second array, so as to enable collective amounts of the first and second directed disinfecting UV energies to be configured by the corresponding numbers of first and second disinfecting UV engine means, at least in part.

The first array may include a plurality of first disinfecting UV engine means which may be spaced at a first spacing, and/or the second array may include a plurality of second disinfecting UV engine means which may be spaced at a second spacing, wherein the first spacing may be greater than, equal to, or less than the second spacing, and/or wherein each of the disinfecting UV engine means of the first or second arrays may be respectively interspersed between a pair of disinfecting UV engine means of the second or first arrays.

Another aspect may provide a linear disinfection device for controllably emitting and directing disinfecting UV energy to at least one first proximal target location which may be defined in a first plane, and at least one second distal target location which may be defined in a second plane, in a user space, comprising a linear housing means mountable on or in at least one support means and having an outer boundary means, the linear housing means containing a first array of first disinfecting UV engine means configured to deliver a first directed disinfecting UV energy to the at least one first proximal target location, and a second array of second disinfecting UV engine means configured to deliver a second directed disinfecting UV energy to the at least one second proximal target location, wherein the outer boundary means is configured to provide two or more common output means extending along the linear housing means, each of the two or more common output means configured to receive both the first directed disinfecting UV energy from the first array of first disinfecting UV engine means and receive the second directed disinfecting UV energy from the second array of second disinfecting UV engine means to deliver the first and second directed disinfecting UV energies to the first and second target locations.

One or each of the first and second disinfecting UV engine means may include a corresponding first and second UV energy director means, wherein one or each of which may receive UV energy from a corresponding first and second UV energy emitter means.

One or each of the first and second UV energy director means may include an optical coupling means between a corresponding first and/or second UV energy diffuser means, a corresponding first and/or second UV energy collimator means, and/or the corresponding first and/or second UV energy emitter means.

Instances of the first disinfecting UV engine means in the first array may be fewer in number than instances of the second disinfecting UV engine means in the second array, so as to enable collective amounts of the first and second directed disinfecting UV energy to be configured by the corresponding numbers of first and second disinfecting UV engine means.

The first disinfecting UV engine means of the first array may be spaced at a first spacing, and/or the second disinfecting UV engine means of the second array may spaced at a second spacing, wherein the first spacing may be greater than the second spacing, and/or wherein each of the first disinfecting UV engine means may be interspersed between a pair of second disinfecting UV engine means.

### BRIEF DESCRIPTION OF THE FIGURES

Several exemplary embodiments of the present disclosure will be provided, by way of examples only, with reference to the appended drawings, wherein:

FIG. 1 is perspective view of a disinfection device shown schematically in an operable configuration showing a housing structure attached to and removed from a support structure;

FIG. 2 is a perspective assembly view of the disinfection device of FIG. 1 with the housing structure disengaged from the support structure;

FIG. 3 is a side assembly view and FIG. 6 is a side schematic view of the disinfection device of FIG. 2;

FIGS. 4, 5 and 6 are schematic cross sectional views of the disinfection device taken on lines 4-4, 5-5 and 6-6 respectively of FIG. 2;

FIGS. 7 to 9 are front and rear perspective views of another disinfection device;

FIG. 10 is a right hand side elevation of the device of FIG. 7, with the left hand side elevation being a mirror thereof and omitted;

FIG. 11 is a part assembly perspective sectional view taken along the break line of FIG. 7;

FIG. 12 is another sectional view according to FIG. 11 schematically illustrating first and second two target locations;

FIG. 12a is a magnified fragmentary part assembly perspective sectional view of a portion of the device shown in FIG. 11;

FIG. 13 is a perspective view of a housing structure segment of the device of FIG. 7;

FIG. 14 is a perspective sectional view of the housing structure segment taken on line 14-14 of FIG. 21;

FIG. 15 is a perspective sectional view of the housing structure segment taken through the break line of FIG. 14;

FIGS. 16, 17, 18 and 19 are top plan, bottom plan, front elevation and rear elevation views of the device of FIG. 7;

FIGS. 20 and 21 are upper front perspective and lower rear perspective views of the housing structure segment of the device of FIG. 13;

FIGS. 22, 23, 24 and 25 are top plan, bottom plan, front elevation and rear elevation views of the housing structure segment of the device of FIG. 13;

FIGS. 26, 27 and 28, are upper right front perspective, lower rear perspective, and upper left front perspective views of a support structure of the device of FIG. 7; and

FIGS. 29, 30, 31 and 32 are top plan, front elevation, bottom plan, and rear elevation views of the support structure of FIG. 26.

FIGS. 33 and 34 are schematic perspective operational views showing disinfecting UV energy to a target surface; and

FIGS. 35 and 36 are plan views of respective target surfaces with plotted values representing disinfecting UV energy strength across a plurality of sites thereon.

The broken lines used in the figures are provided for purposes of illustrating portions of the design that include environment. Further, the subject matter herein includes any of the present figures with any solid line therein replaced by a broken or stippled line, or vice versa. Thus, any such replacement is not new matter.

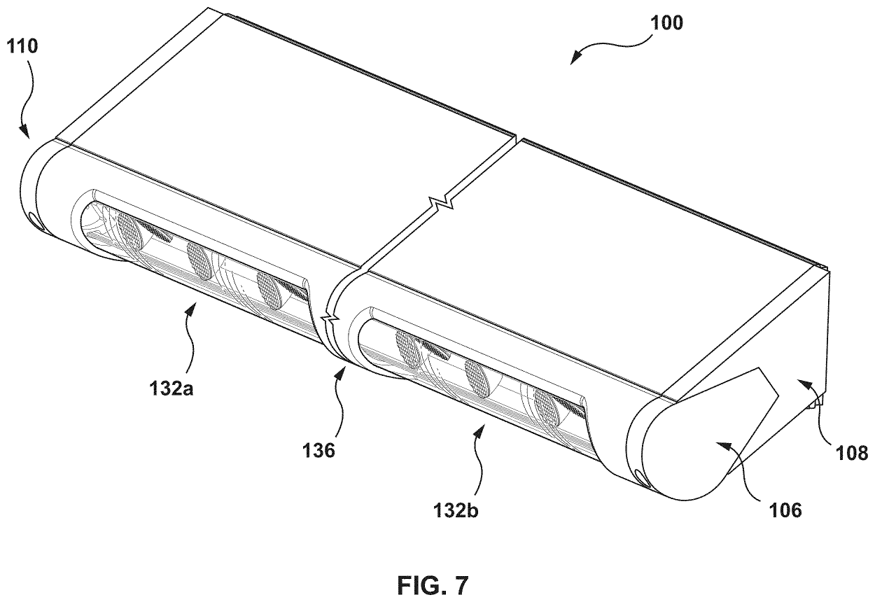

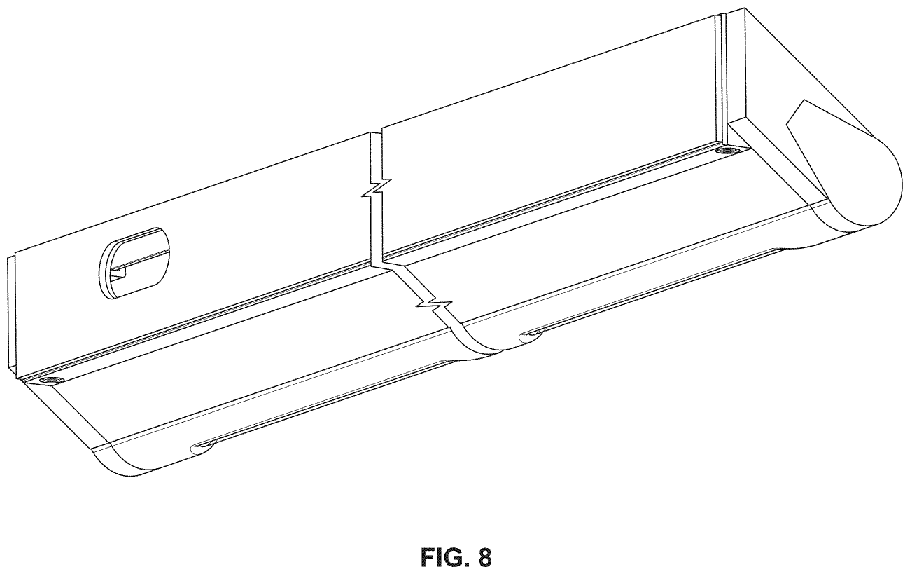

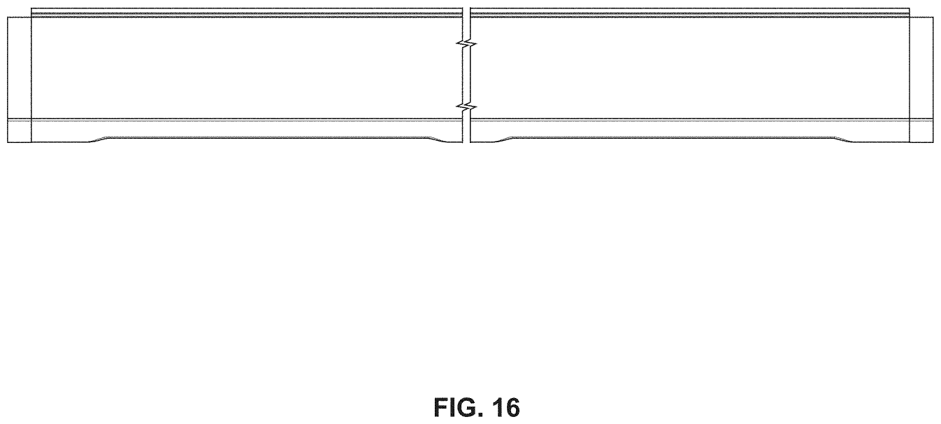

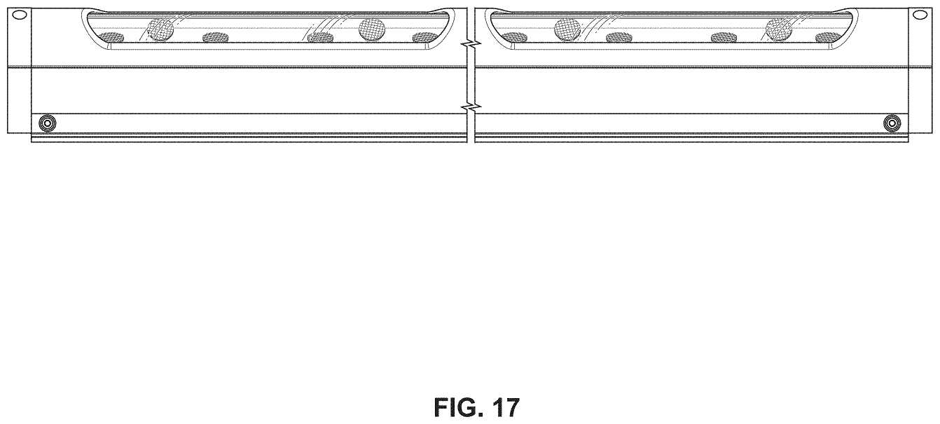

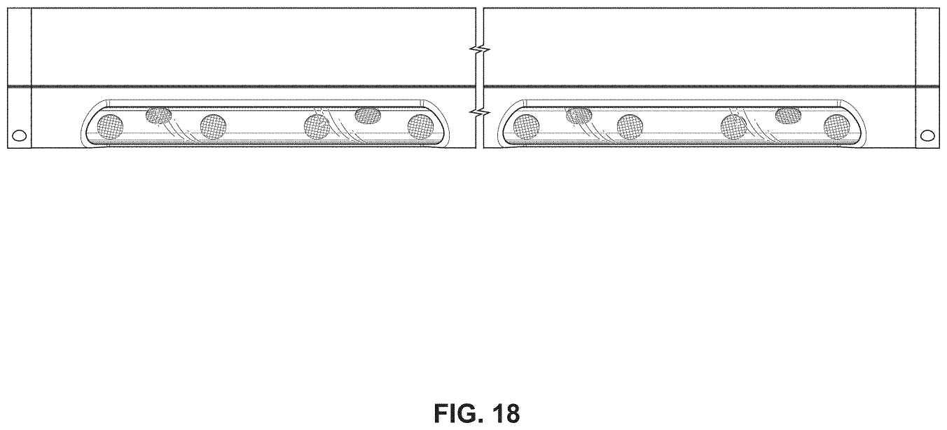

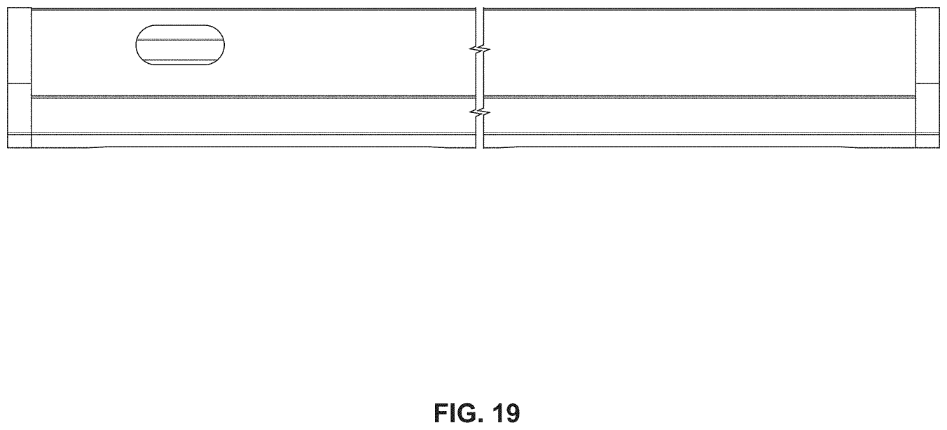

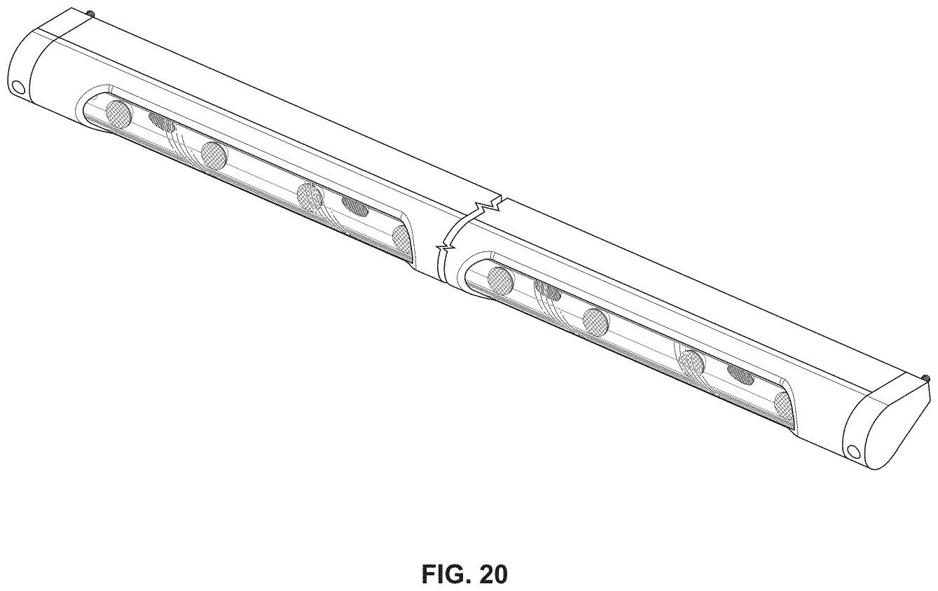

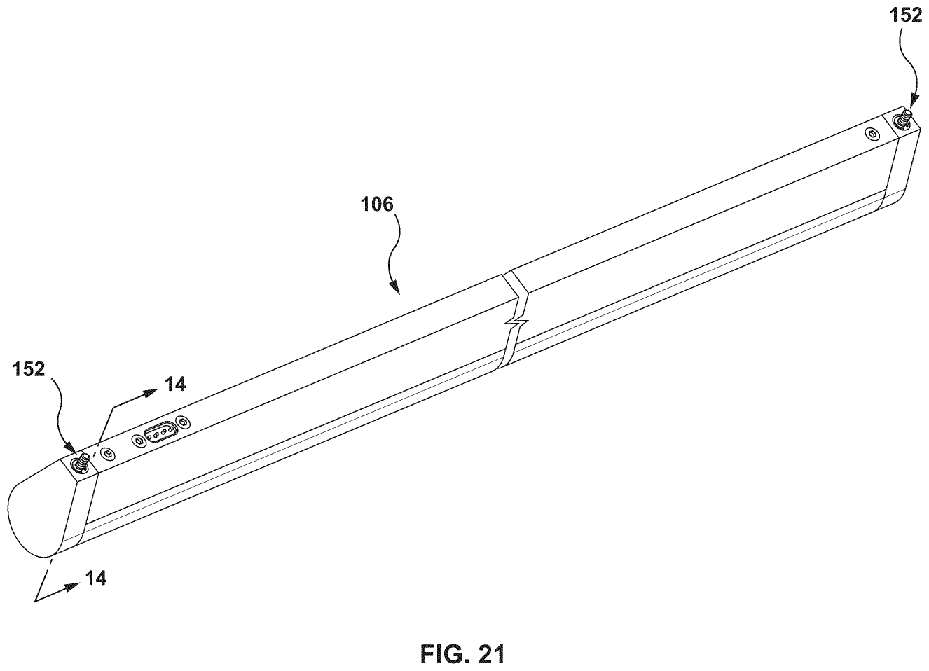

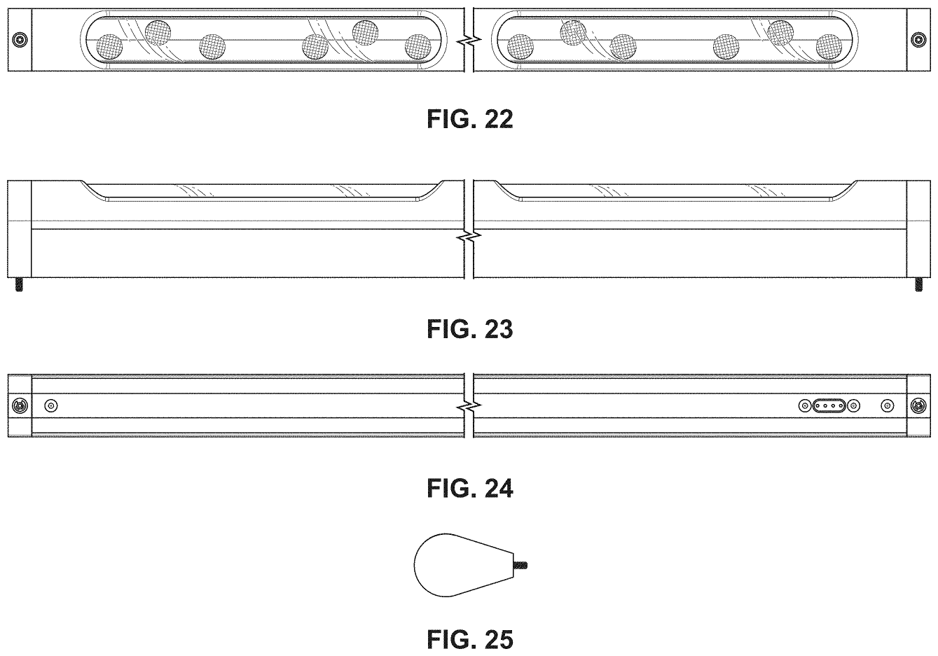

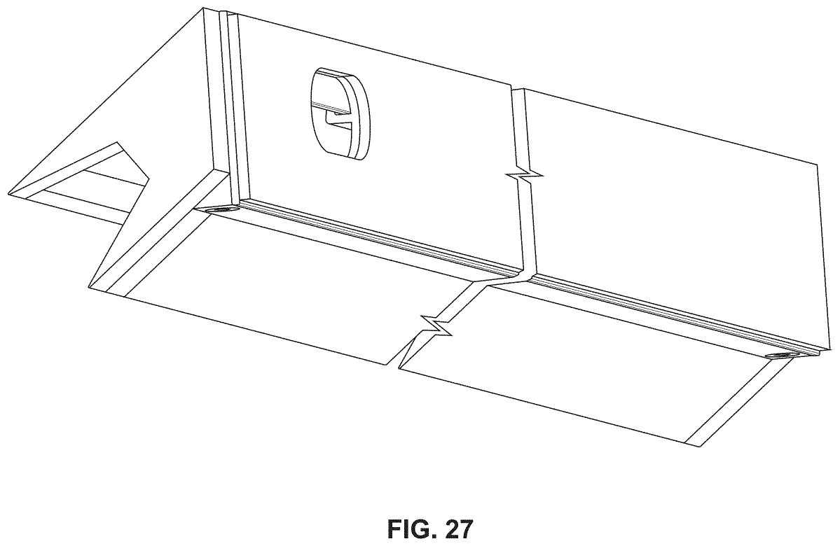

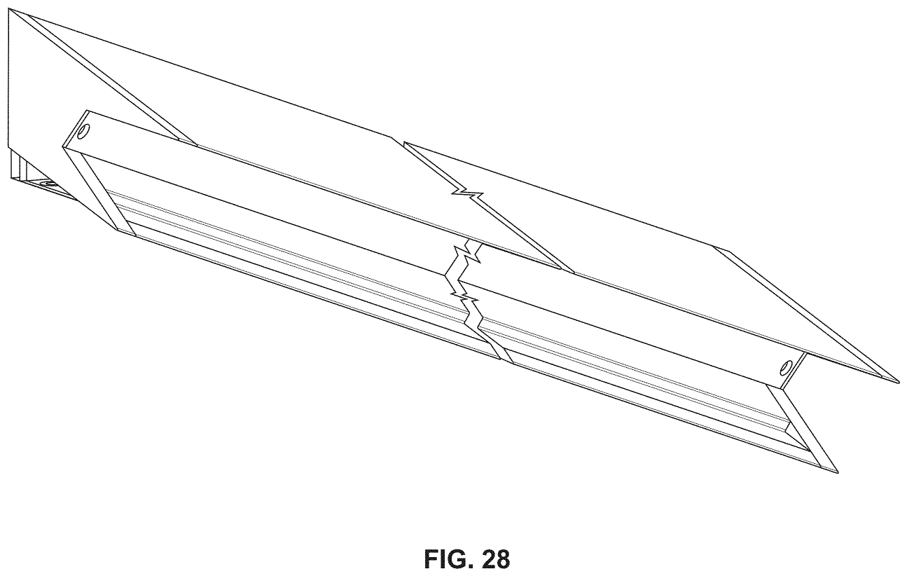

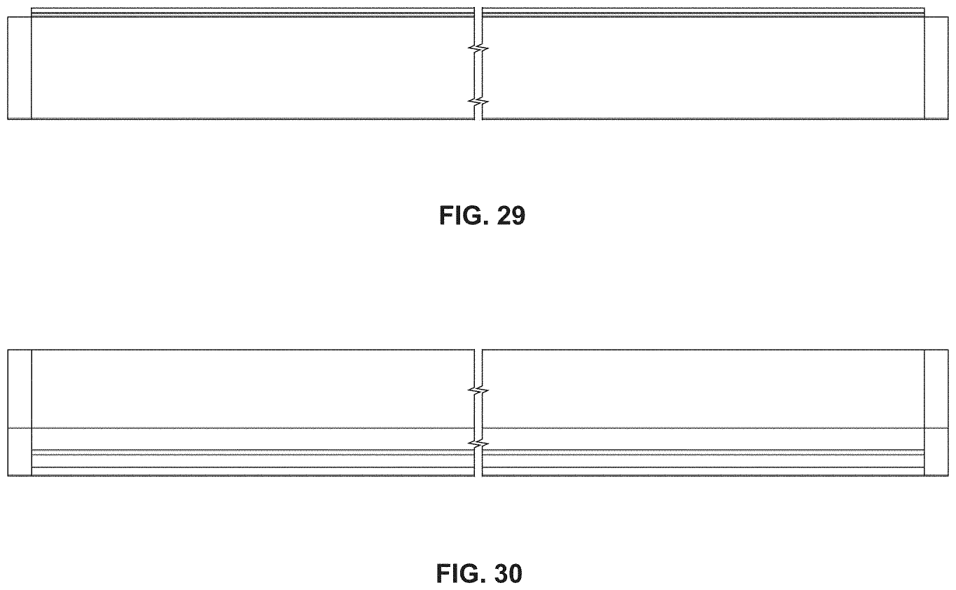

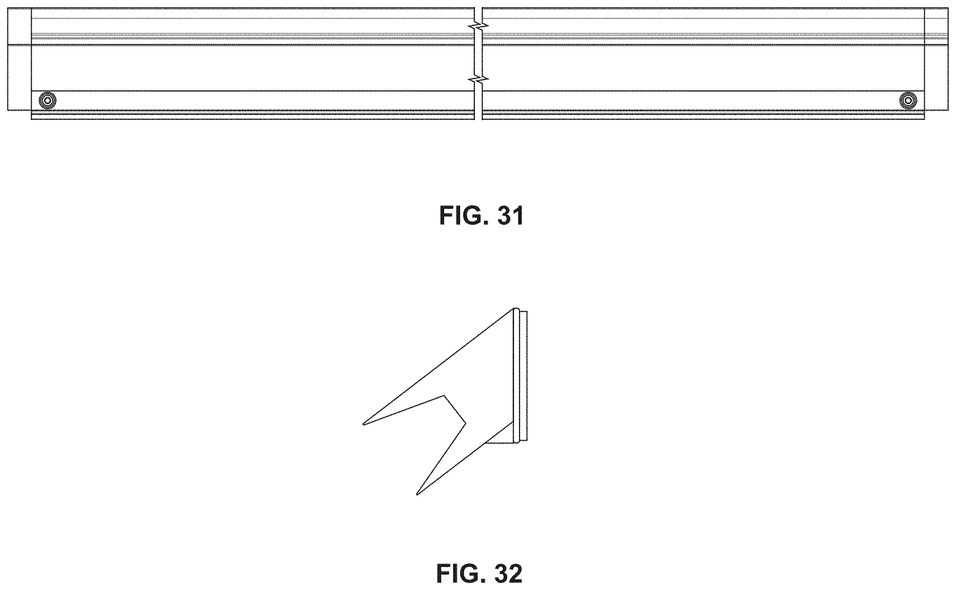

The disinfection device is shown in FIGS. 7-9, 13, 14, 16-24 and 26-31, with symbolic break lines along its length. The appearance of a portion of the luminaire structure between the break lines does not form part of the design. Any specific dimension of the article associated with the break lines is not claimed and is to be broadly interpreted. The subject matter herein includes any of the present figures with any such symbolic break lines removed, or with one or more further symbolic break lines added. Thus, any removal or addition of one or more any such symbolic break lines is not new matter. Further, the subject matter herein includes any of the present figures with any solid line therein replaced by a broken or stippled line, or vice versa.

### DETAILED DESCRIPTION

It should be understood that the invention is not limited in its application to the details of construction and the arrangement of components set forth in the following description or illustrated in the drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical, mechanical or other connections or couplings. The terms upper, lower, and vertical are intended for operative context only and are not necessarily intended to limit the invention only to those configurations or orientations. In any instance in which the disclosure refers to a single instance of an element, example embodiments may include a multiple of such elements. The term “at least one” in reference to any element is not intended to force an interpretation on any other reference elsewhere in the disclosure to a single instance of an element to mean only one such instance of the element. Furthermore, and as described in subsequent paragraphs, the specific mechanical and/or other configurations illustrated in the drawings are intended to exemplify embodiments of the invention. However, other alternative mechanical and/or other configurations are possible which are considered to be within the teachings of the instant disclosure. Furthermore, any one element, feature, structure, function, of any aspect and/or example embodiment described in the present disclosure including the figures, clauses and/or claims herein, may itself be claimed on its own or be combined with any one or more elements, features, structures, functions, and/or steps from the same or any other aspects and/or example embodiments described in the present disclosure including the figures, clauses and/or claims herein.

The term “UV LED” or variations thereof used herein may include, but not be limited to, semiconductor, electroluminescent, organic, polymeric or other material based ultraviolet energy sources, such as those which commonly known as generally as light emitting diodes, as well as polymeric and other such energy sources, equivalents and variants thereof, which emit energy at frequencies known to kill at least some viruses, bacteria and/or other pathogens where such pathogens have been made available for testing. The UV LED may be configured to emit UV energy in the range of frequencies including but not strictly bounded by UV-C: 200 to 280 nm: UV-B: 280 to 315 nm and UV-A: 315 to 400 nm.

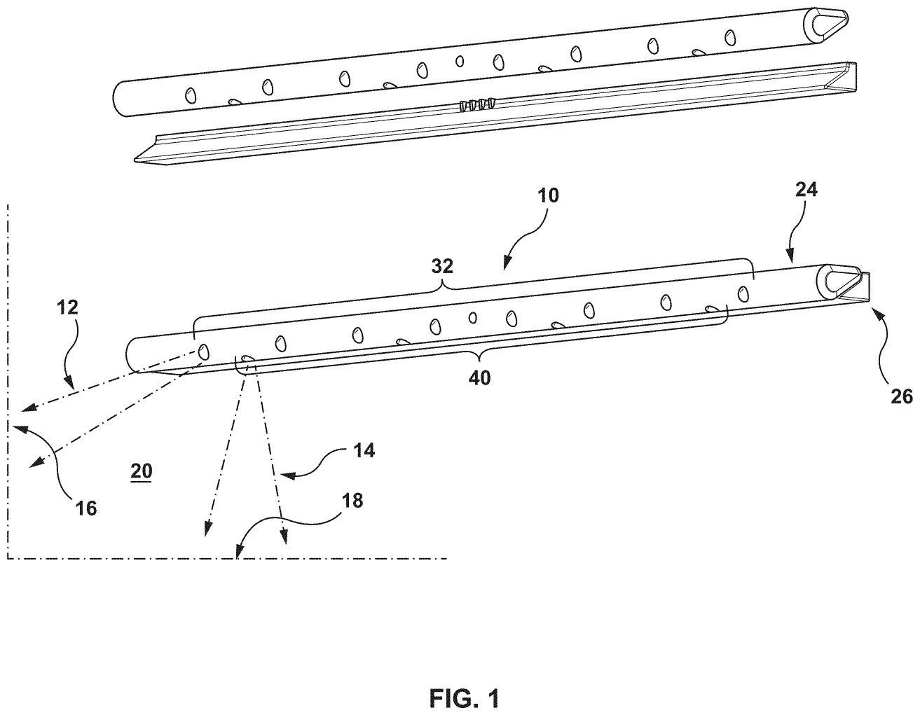

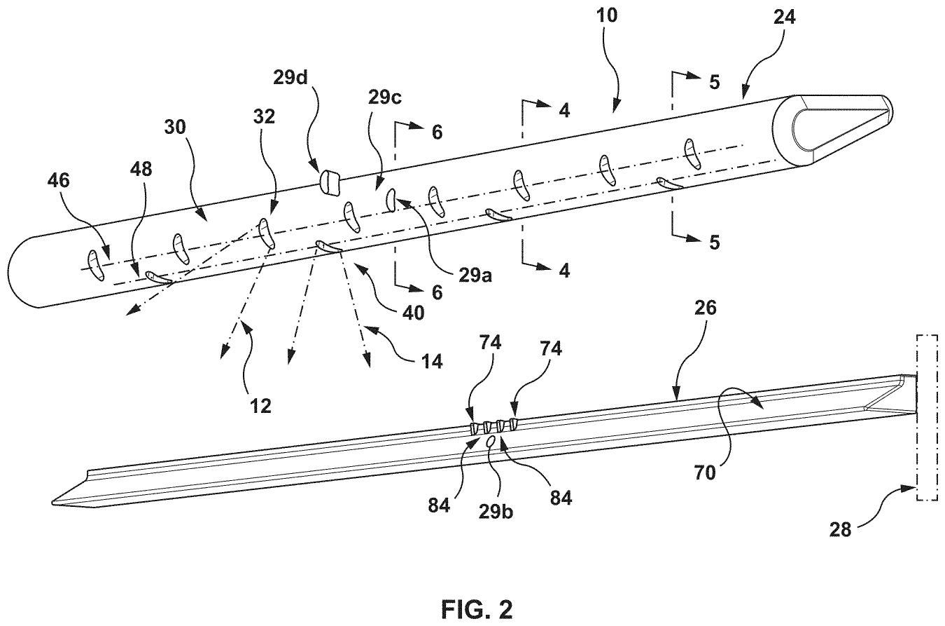

Referring to the FIG. 1 and subsequent figures, there is provided a disinfection device 10 for controllably emitting and directing UV energy, such as shown at 12, 14, to at least one target location, such as first target location 16, which can be an elongate first linear target location 16 and at least one second target location, such as second target location 18 which can be an elongate second linear location 18 at one or more designated common sites in an interior (or exterior) user space 20 such as a a sink, the walls, counter, and other surfaces surrounding and/or near the sink area as one example. The disinfection device 10 has a linear housing structure, which can be a linear housing structure 24 mountable on at least one support structure, which can be a linear support structure 26, which may be provided with at least one surface for abutting an appropriate mounting surface 28 (FIG. 2), such as on a lower surface of a mirror, or above a key pad on a kiosk, among other possible contact surfaces in user spaces.

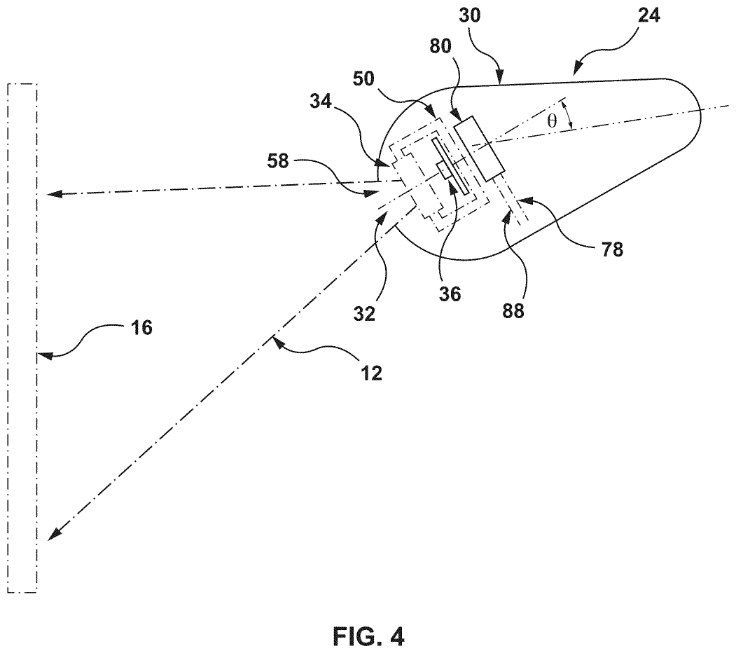

As shown in FIG. 4, the disinfection device 10 may be provided with an outer boundary 30 that may be configured to provide a first array of outputs shown at 32 in a generally linear orientation of first linear array of outputs 32 to deliver directed UV energy 12 at a first angle θ, relative to a reference axis of the linear housing structure 24, to the at least one first linear target location 16. Each output of the first array 32 may be configured to receive directed UV energy from one instance of a first array of UV energy directors, or first directors, shown at 34, such as a lens or other focusing or directing optic. Each first director of the first array 34 may be configured to receive UV energy from one instance of a first array of UV LEDs 36.

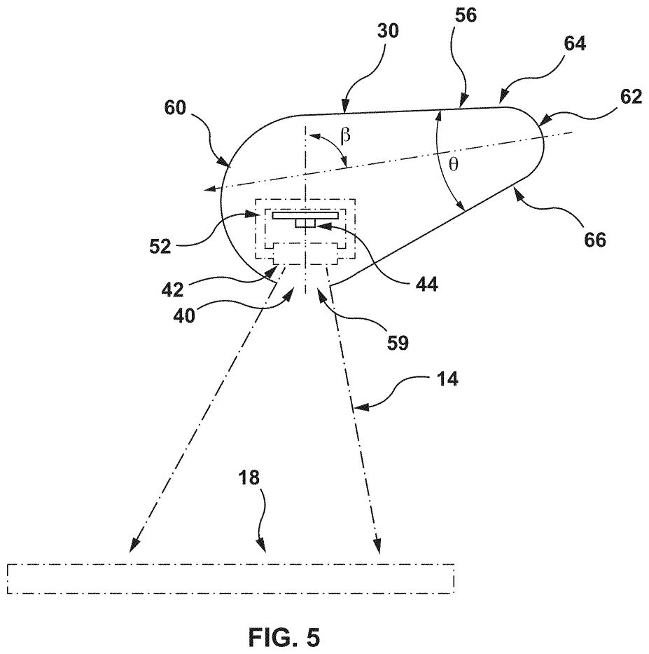

Referring to FIG. 5, the outer boundary 30 may be configured to provide a second array of outputs 40 in a generally linear orientation of second linear array of outputs 40 to deliver directed UV energy at a second angle β to the at least one second linear target location 18. Each output of the second array 40 may be configured to receive directed UV energy from one instance of a second array of UV energy directors, or second directors, shown at 42 in FIG. 5. Each UV energy director of the second array 42 may be configured to receive UV energy from one instance of a second array of UV LEDs 44.

Referring to FIG. 2, the first array 32 and second array 40 can be seen to extend along respective parallel axes 46, 48 and such axes 46, 48 may be substantially parallel or substantially in common.

Referring to FIGS. 4 and 5, each instance of the UV energy directors of the first array 34, and the corresponding instance of the UV LEDs of the first array 36 may be positioned at a first mounting structure 50, such as a linear mounting structure 50, in the linear housing structure 24. Meanwhile, each instance of the UV energy directors of the second array 42, and the corresponding instance of the UV LEDs of the second array 44 may be positioned at a second mounting structure 52, such as a linear mounting structure 52, in the linear housing structure 24.

The outer boundary 30 of the linear housing structure 24 may be defined by an outer boundary wall 56, with each incidence of the first and second arrays of outputs 32 and 40 provided by corresponding passages 58, 59 through the boundary wall 56. The outer boundary wall 56 may have a continuous or discontinuous cross section. The outer boundary wall 56 may, for example, alternatively or in addition, have a curved, linear and/or curvilinear cross section.

Referring to FIG. 5, the outer boundary wall 56 may be provided with ellipsoidal (or circular) end regions 60, 62, with equal or unequal radii, with opposed wall sections extending therebetween, which may be oriented with an acute included angle α.

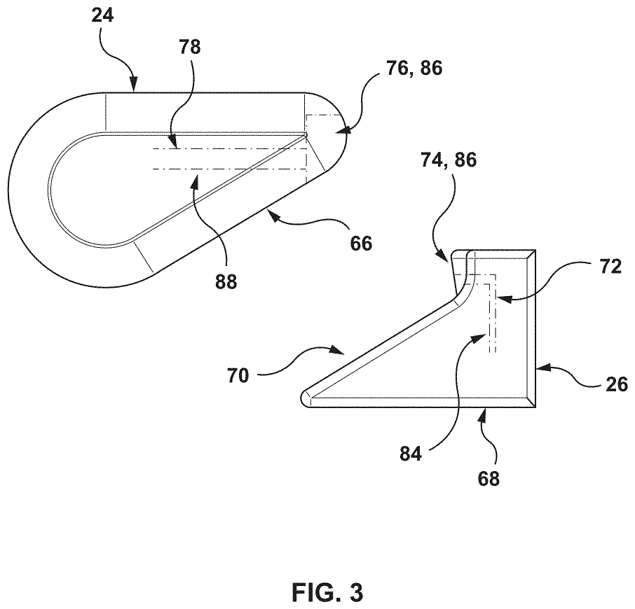

Referring to FIG. 3, the support structure 26 may include a base 68 with a support surface 70, which for illustration purposes may be seen to be inclined relative to the mounting structure 28 as seen in FIGS. 2 and 3, to engage a corresponding wall section 66.

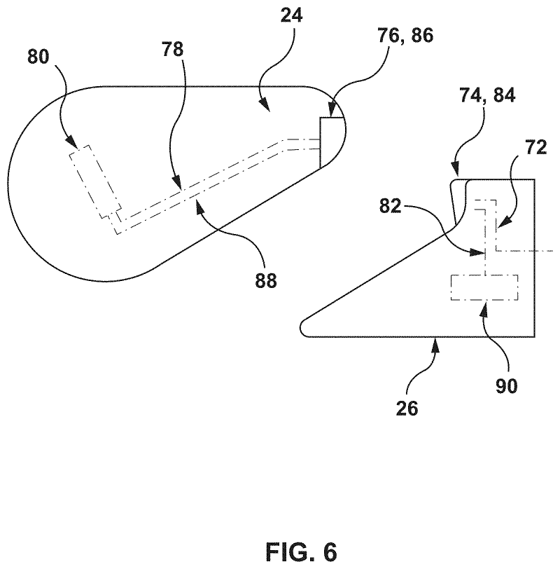

The support structure 26 may be further provided with a first power supply segment 72, while the linear housing structure 24 and support structure 26 are each configured with a first set of contacts 76, 74, respectively, for coupling the first power supply segment 72 to a second power supply segment 78 in the linear housing structure to energize the first and second arrays of UV energy emitters 36, 44. The second power supply segment 78 may include or be in communication with at least one driver 80, as shown in FIG. 4, to drive the UV LEDs, and which may be configured to exchange control data signals with a controller 90 (FIG. 6) networked therewith. In this case, while the driver 80 is shown in FIG. 4, as positioned adjacent the first linear mounting structure 50 and the first array of UV LEDs 36, the driver 80 may be configured to power both the first and second arrays of UV LEDs 36, 44.

Referring to FIG. 6, the support structure 26 may further include, as shown schematically, a first control data path segment 82, while the linear housing structure 24 and support structure 26 are each configured with a second set of contacts 84, 86 for coupling the first control data path segment 82 to a second control data path segment 88 in the linear housing structure 24 in communication with the driver 80. In this case, the controller 90 may be provided in the support structure 26, the linear housing structure 24, or be a remote controller located at a remote location.

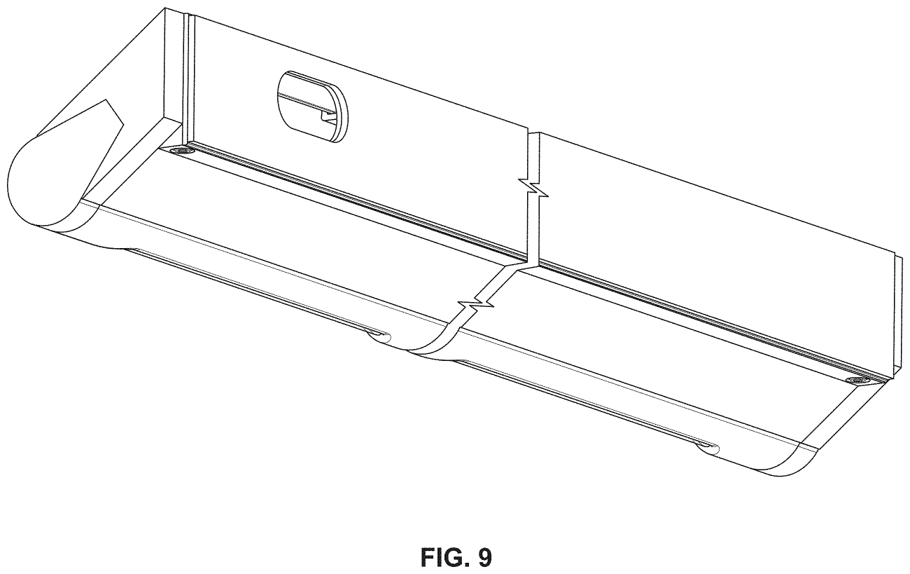

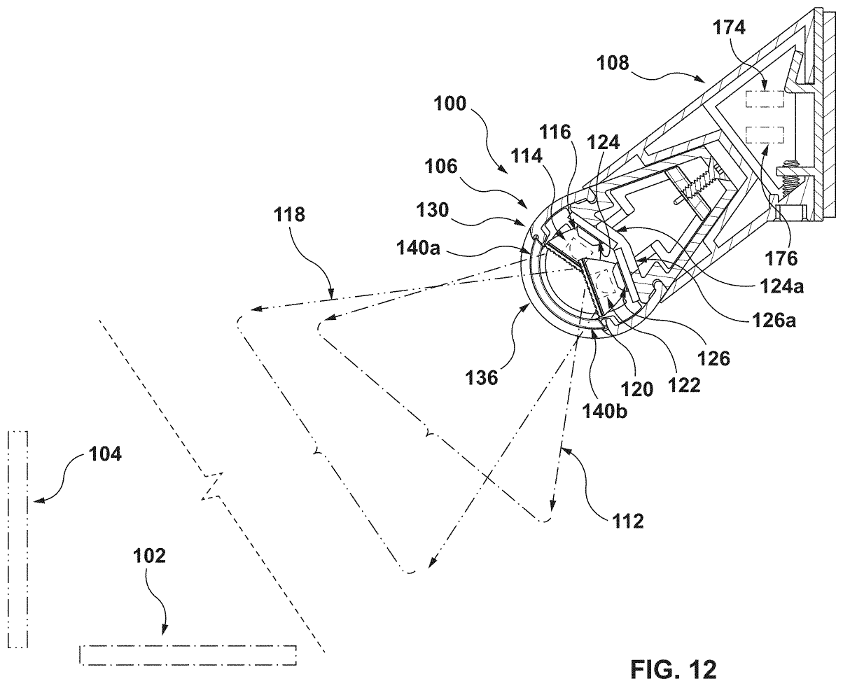

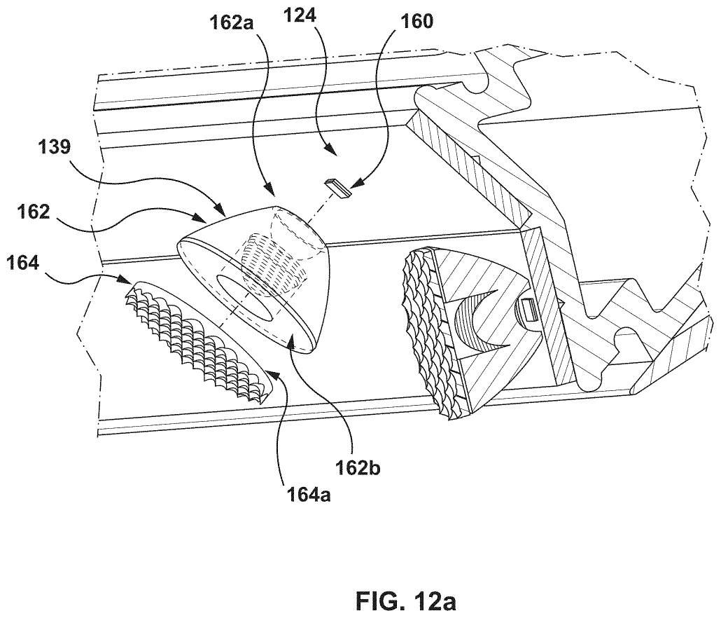

Another example embodiment of a disinfection device is shown at 100 in FIGS. 7 to 32 for controllably emitting and directing disinfecting UV energy to at least one first target location 102 (FIG. 12), which may be a linear first target location, and at least one second target location 104, which may be a linear second target location, in a user space. Referring to FIG. 7, the device 100 has a linear housing structure 106 mountable on or in at least one support structure 108 and has an outer boundary 110.

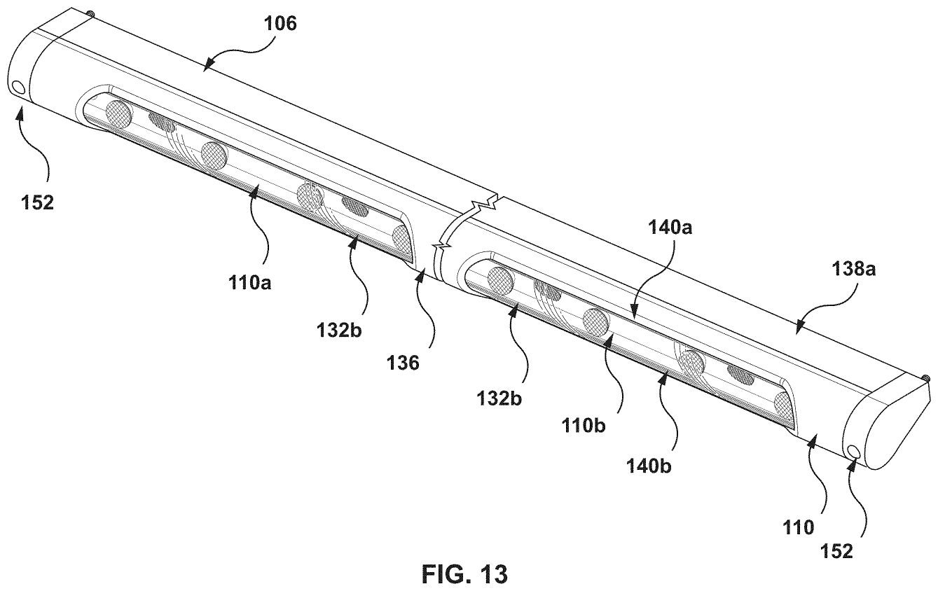

The linear housing structure 106 may be decoupled from the support structure 108, as shown in FIG. 13. The outer boundary 110 may be configured to provide at least one, in this case a pair of outputs 110a, 110b, which as shown in FIG. 13, may both be configured to receive a first directed disinfecting UV energy 112 (FIG. 12) from at least one instance of a first array of disinfecting UV energy directors shown at 114. Each UV energy disinfecting director 114 of the first array thereof may be configured to receive a first disinfecting UV energy from one instance of a corresponding first array of disinfecting UV energy emitters (in this example UV LEDs) shown at 116. The outputs 110a, 110b may thus be configured to deliver the first directed disinfecting UV energy 112 to the at least one first target location 102.

Likewise, both outputs 110a, 110b may be configured to receive a second directed disinfecting UV energy 118 from at least one instance of a second array of disinfecting UV energy directors shown at 120. Each disinfecting UV energy director 120 of the second array may be configured to receive a second disinfecting UV energy from one instance of a second array of disinfecting UV energy emitters (in this example UV LEDs) shown at 122. The outputs 110a, 110b may thus be configured to deliver the second directed disinfecting UV energy 118 to the at least one second linear target location 104. The first and second arrays, the housing structure and the support structures may be linear or nonlinear, while the housing structure may include more arrays as needed to as to provide a multiple of arrays for the delivery of UV energy to at least the first and second target locations.

Referring to FIG. 12, each instance of the disinfecting UV energy directors 114 of the first array thereof, and the corresponding instance of the disinfecting UV energy emitters 116 of the first array thereof may be positioned adjacent a common location on a first linear mounting surface 124 in the linear housing structure 106.

As well, each instance of the disinfecting UV energy directors 120 of the second array thereof, and the corresponding instance of the disinfecting UV energy emitters 122 of the second array thereof may be positioned adjacent a common location on a second linear mounting surface 126 in the linear housing structure 106.

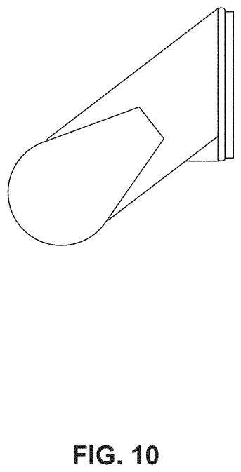

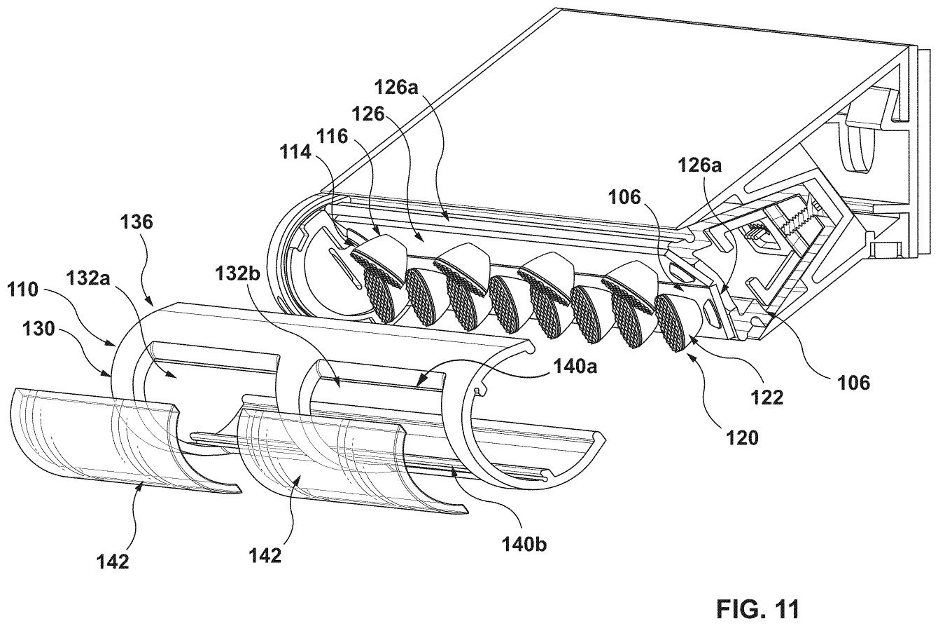

The first and second linear surfaces 124, 126 may be defined by corresponding first and second linear LED board structures 124a, 126a positioned in the linear housing structure 106. Referring to FIG. 11, the number of first UV energy directors 114 and first UV energy emitters 116 along the first linear mounting surface 124 may be fewer in number, than the number of second UV energy directors 120 and UV energy emitters 122 along the second linear mounting surface 126 (or vice versa), which enables a collective amount first directed disinfecting UV energy 112 delivered to the first target location 102 to be less than a collective amount second directed disinfecting UV energy 118 delivered to the second target location 104 (or vice versa).

Referring to FIG. 11, the outer boundary 110 of the linear housing structure 106 may be defined by an outer boundary wall 130, with each output 110a including a passage 132a, 132b through the boundary wall 110.

Referring to FIGS. 11, 13 and 14, the outer boundary wall 110 may be provided with at least one ellipsoidal end region 136, with a pair of opposed wall sections 138a, 138b extending therefrom. In this case, each passage 132a, 132b may extend through the at least one ellipsoidal end region 136 along a dimension substantially parallel to a central axis thereof.

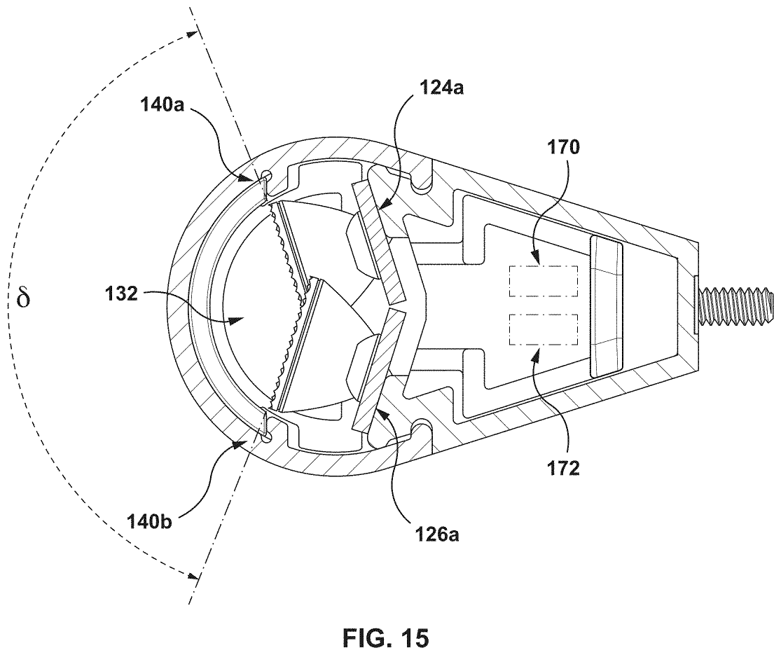

Referring to FIGS. 13 and 15, each passage 132a, 132b may be defined by a pair of opposed linear boundaries 140a, 140b that are spaced by an included radial angle δ, such as for example between about 30 degrees and about 180 degrees, or between about 90 degrees and about 175 degrees, for example about 170 degrees, which may be selected according to a particular application, with corresponding configurations made to the orientation and optical characteristics of the first UV energy directors, 114 and first UV energy emitters 116, and second UV energy directors 120 and second UV energy emitters 122.

In the case of FIG. 11, the first array of UV LEDs 116 can be seen to be interspersed between a corresponding pair of instances of the second array of UV LEDs 122 but alternate in such a way that there are alternating gaps where no such instance of the first array of UV LEDs 116 is provided. This may enable collective amounts of first and second directed disinfecting UV energy 112, 118, to be configured by the allocation of first UV energy directors and emitters 114, 116 and second UV energy directors and emitters 120, 122 as the case may be. For instance, different groupings of one or more UV energy directors and emitters may be provided in different combinations in the first and second arrays depending on the desired first and second directed disinfecting UV energies 112, 118. For instance, in a disinfecting device for use in a bathroom, mounted above a sink, a central grouping of second UV energy directors and emitters 120, 122 may be provided to deliver a greater level of the second directed disinfecting UV energy to a target location occupied by a faucet and facet knobs, compared with other neighboring areas, which may receive a substantially equivalent amount of second directed disinfecting UV energy as a first directed disinfecting UV energy being delivered to a wall surface above the sink.

In some example embodiments, the one or more groupings of second UV energy directors and second UV energy emitters 120, 122 in the second array may be higher in number, for example as shown in the example of FIG. 11, to take into account the greater distance of travel to the sink, while the one or more groupings of first UV energy directors and first UV energy emitters 114, 116 in the first array may be lower in number, for example as shown in the example of FIG. 11, to take into account the shorter distance of travel to a wall surface above the sink.

FIG. 12a shows a magnified view of UV engines 139 providing the first and second UV energy emitters 116 and 122 by way of UV LEDs 160, and the first and second UV energy directors 114 and 120 provided by a collimator structure 162 with a surface 162a to engage the first surface and second surfaces 124, 126, and a diffuser structure 164 with a surface 164a to engage a corresponding surface 162b on the collimator structure 162.

Referring to FIG. 12, the first and second linear LED board structures 124a, 124b may each be configured to extend adjacent or along a corresponding linear boundary 140a, 140b, so as to cause to the first disinfecting UV energy 112 of the first array to cross or pass through the second directed disinfecting UV energy 118 of the second array (and vice versa).

Referring to FIG. 11, the disinfection device 100 may be further provided with a UV energy transmissive cover structure 142 to close each passage 132a, 132b. The cover structures 142 can be formed of UV energy transmissive materials which may include without limitation quartz and other materials or combinations thereof. In some example embodiments, the end region 136 may be planar or rectilinear, at least in part, to provide passages 132a, 132b such that they can receive cover structures that may be flat, rectilinear and/or planar at least in part.

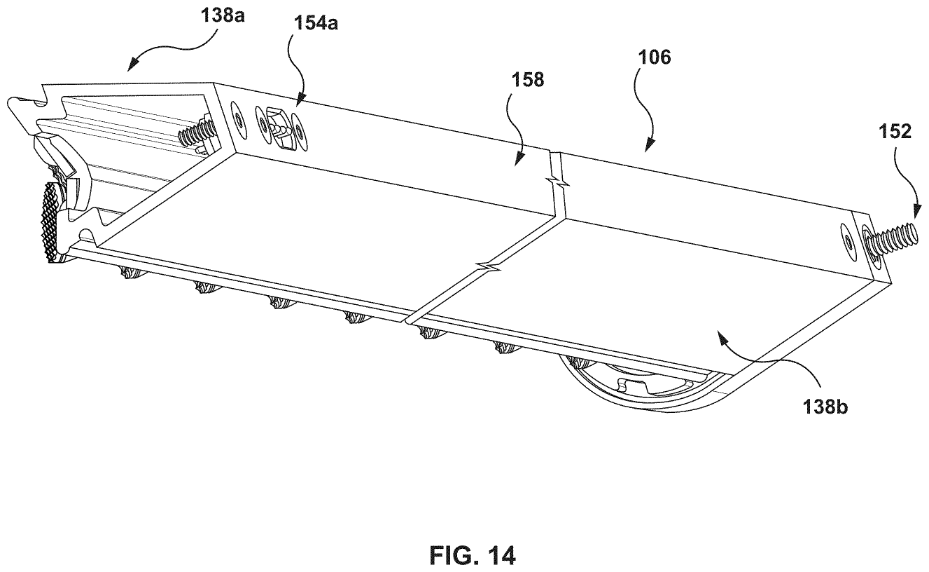

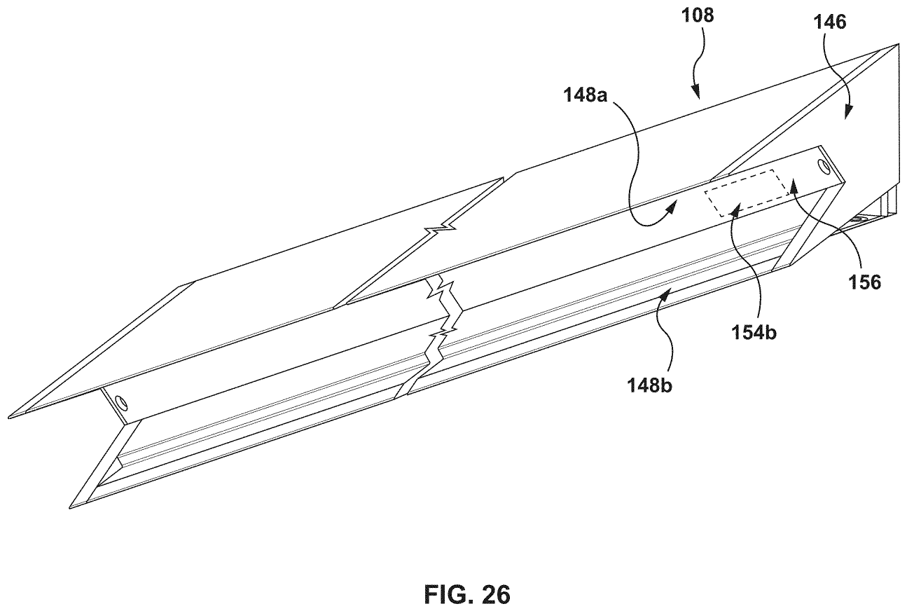

As can be seen in FIGS. 14 and 26, the support structure 108 may include a base structure 146 with at least one support surface 148b, such as an inclined support surface 148b, to engage the corresponding at least one wall section 138b on the linear housing structure 106. In this case, the base structure 146 may further provide a pair of opposed support surfaces 148a, 148b to engage the corresponding wall sections 138a, 138b, so as to position the linear housing structure into the support structure, for example in a wedge-like configuration, with at least one, in this case a pair of anchor structures shown generally at 152 adjacent opposite end regions of the device 100, to anchor the linear housing structure 106 in the support structure 108 in the wedge-like configuration. Thus, one or more of the surfaces 136a, 148a and the surfaces 128b, 148b may provide an effective heat transfer interface for dissipation of heat from the housing structure 106 generated by the first and second arrays of UV LEDs 116, 122, and associated heat generating structures and components therein. In this case, the wedge-like configuration under the forces of anchor structures 52 may provide improved heat transfer in some cases in the heat transfer interface therebetween.

As can be seen in FIG. 14 and schematically in FIG. 26, the linear housing structure 106 and the support structure 108 may further comprise complementary communication nodes 154a, 154b for transferring power and/or communication signals therebetween. For example, the support structure 108 and the linear housing structure 106 may include respective adjacent surfaces 156, 158 in the wedge like configuration with the communication pathways defined thereon. One or more configurations may be provided to enable the device 100, or other devices, methods, processes or systems disclosed herein, to be enabled to be automatic, autonomous or otherwise responsive to data representative of one or more changes in conditions in the user space to initiate a change of state of the device, for example by enabling or disabling certain functions, in one example instance by enabling or disabling one or more of the disinfecting UV engines.

Referring to FIGS. 12 and 15, the linear housing structure 106 may be provided with data storage structure shown schematically at 170 to receive and store data such as authentication data identifying one or more parameters of the linear housing structure, such as a date of fabrication, a change of status of a fault code indicating improper or faulty operation of any of the first or second UV energy emitters, which may be updated by a monitor 172. Similarly, the support structure 108 may be provided with authenticator 174 and a power controller 176, wherein the authenticator may be configured to direct the power controller to deliver power to the first and second arrays when at least one parameter meets a first designated condition, or to interrupt power when at least one parameter meets a second designated condition.

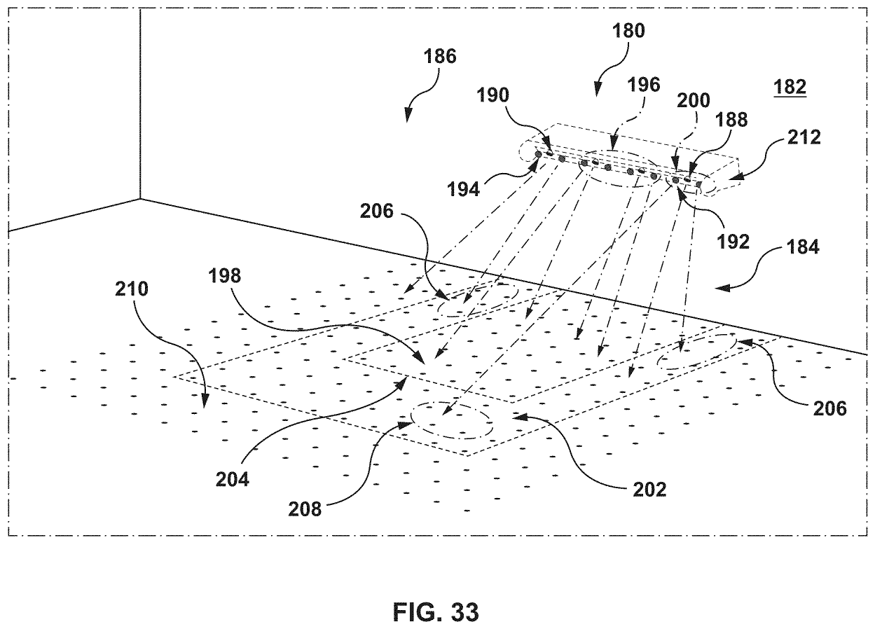

Referring to FIG. 33, there is provided a disinfection device shown at 180 which is mounted on a wall surface 182 and shown in an operative mode for controllably emitting and directing respectively trained disinfecting UV energy 184 to a plurality of regions in an interior or exterior user space 186. The device 180 may be in the form of any of the example embodiments herein, and any individual component, feature or function thereof, alone or in combination. In this example, the disinfection device 180 is provided with a first array, shown schematically at 188, of at least one first disinfecting UV engine 190 configured to deliver a first directed disinfecting UV energy, and a second array 192 of at least one second disinfecting UV engine 194 configured to deliver a second directed disinfecting UV energy.

In this case at least one first group shown at 196 of the first and/or second disinfecting UV engines 190, 194 may be configured to deliver the first and/or second disinfecting energies to the at least one first target region 198, and at least one second group 200 of the first and/or second disinfecting UV engines 190, 194 may be configured to deliver the first and/or second disinfecting energies to at least one second target region 202. In this example, the first target region 198 is centrally located relative to the second target region 202 which surrounds the central region on a number of boundaries thereof, in this case three boundaries on one or more common planes, at least in part.

The first and second groups 196, 200 may be configured to form at least one disinfecting UV energy transition 204 along one or more of the boundaries, between the first and second target regions 198, 202, with each of the first and second disinfecting UV energies in the respective groups at a designated energy or luminance strength according to a corresponding attribute of the first and second regions respectively.

Each disinfecting UV engine 190 of the first array 188 may be configured to deliver the first directed disinfecting UV energy to a first proximal field, for example as shown at 206, and each disinfecting UV engine 194 of the second array 192 may be configured to deliver the second directed disinfecting UV energy to a second distal field, for example as shown at 208. For example, each of the first target region 198 and second target region 202 may include at least one first proximal field 206 and/or at least one distal second distal field 208.

The at least one first group 196 may be oriented to deliver the first and/or second directed disinfecting UV energies to the first region 198, which in this example may be a central inner sector on a target surface 210 in the interior user space 182. Meanwhile, the at least one of the second group 200 may be oriented to deliver the first and/or second directed disinfecting UV energy to the second region 208, which in this example may be a peripheral outer sector on the target surface 210 in the interior user space 186.

While the groups 196, 198 are shown to include neighboring disinfecting UV engines of the first and/or second arrays 188, 192, the groups may involve any combination of disinfecting UV engines, whether neighboring and/or adjacent, or not neighboring and/or adjacent. In the case of the second target region 202, it may, as shown in FIG. 33, have peripheral sectors on opposite sides of the central inner section making up the central sector of the first target region 198.

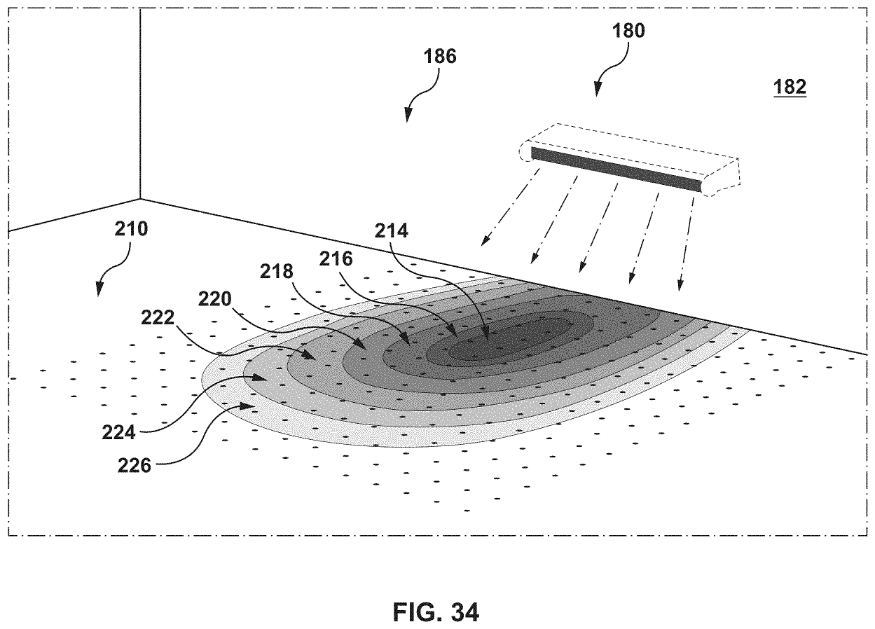

FIG. 34 shows the disinfection device 180 of FIG. 33, and in this example illustrating a number of adjacent and concentric target regions 214 to 226, shown by respective shaded areas, with energy transitions between them, as represented by solid lines between the shaded areas.

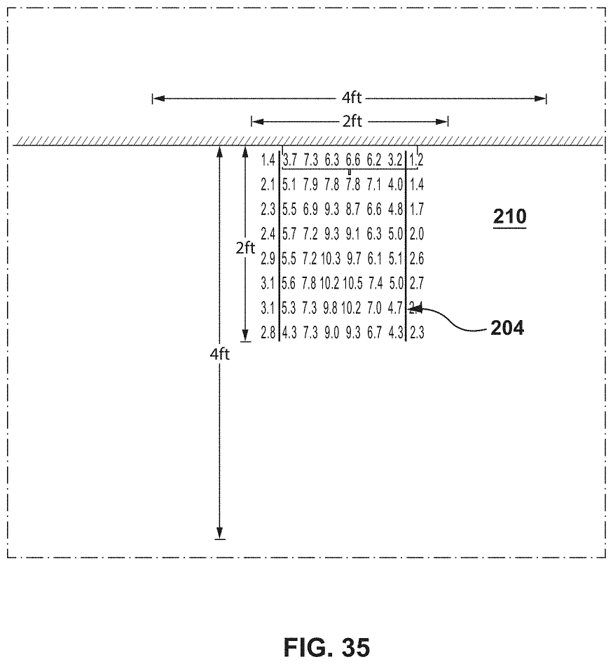

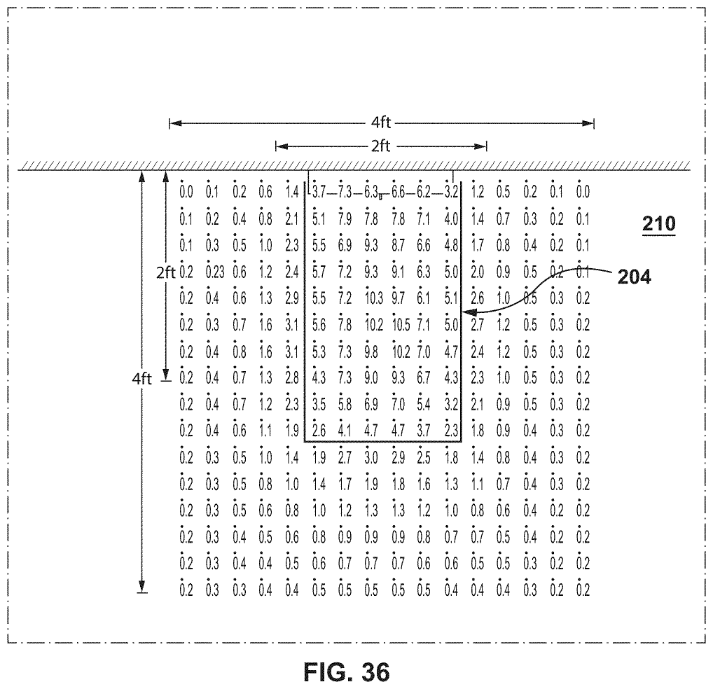

Further, examples of the at least one energy transition 204 may be seen in FIGS. 35 and 36, which illustrate schematic mappings of disinfecting UV energy at each of a grid of designated sites on the target surface 210. In the case of FIG. 36, the transition region 204 may be defined between the adjacent sites which have differences in the disinfecting UV energy according to predetermined, algorithmic, formulaic or probable ranges between them.

The target surface 210 may be a sink, counter, and/or wall surface, at least in part, or for that matter include other surfaces in facilities such as hospitals and other convalescent or care facilities, or in other internal user spaces such as residential and commercial interior or exterior user spaces, in which such surfaces may include walls, floors, doors, lights, furniture, curtains and other barriers, toilets, tubs, showers, wheeled and other assistive devices and the like, and such exterior applicants as ATM (automated teller machines), parking kiosks, building entry panels, or other devices and surfaces as disclosed herein or otherwise known to be susceptible to conditions in which the devices, systems, methods and/or processes herein may be usefully applied.

Referring again to FIG. 33, in some example embodiments, instances of the first disinfecting UV engine 190 in the first array 188 may be equal or unequal, such as fewer, in number compared with the instances of the second disinfecting UV engine 194 in the second array 192, so as to enable collective amounts of the first and second directed disinfecting UV energy to be configured by the corresponding numbers of first and second disinfecting UV engines 190, 194. As with the earlier example embodiments, the first array 188 may include a plurality of first disinfecting UV engines 190 which are spaced at a first spacing, and the second array 192 may include a plurality of second disinfecting UV engines which are spaced at a second spacing, wherein the first spacing is greater than the second spacing, and wherein each of the first disinfecting UV engines 190 is interspersed between a pair of second disinfecting UV engines 194. Further, the disinfecting UV engines may be individually tuned, for example according to the attributes of the intensity of the UV LED 160, and the shape and total internal reflection (TIR) characteristics of the collimator structure 162. Alternatively, the first and/or second disinfecting UV engines 190, 194 may be independently physically oriented or positioned relative to a reference point, line or site, such as may be defined on the target surface 210, the wall surface 182, or an axis, edge, surprise, plane or other structural aspect of a supporting structure such as the housing structure shown at 212.

Thus, in some example embodiments, there may be provided a disinfection device for controllably emitting and directing focused disinfecting UV energy to a plurality of regions in an interior user space. The device may comprise one or more collections of disinfecting UV engines, which may be configured in one or more groups of one or more disinfecting UV energies to deliver focused respective disinfecting UV energies to at least one first target region and at least one second restricted target region, so as to define a transition between the first and second target regions, or between one of the first and second target regions and an adjacent peripheral region beyond the first and or second regions wherein the adjacent peripheral region receives substantially no disinfecting UV energy. Further, in some instances, the transition may be defined by a change in disinfecting UV energy strength of a designated value, according to one or more predetermined, algorithmic, formulaic or probable ranges across the transition.

Clauses

Non-limiting example embodiments are described in the following clauses:

1. A disinfection device for controllably emitting and directing disinfecting UV energy to at least one elongate first target location and at least one second target location in a user space, comprising a housing structure mountable on a support structure and having an outer boundary, wherein the outer boundary is configured to provide:
a first array of outputs to deliver directed disinfecting UV energy to the at least one first target location, with each output of the first array thereof configured to receive directed disinfecting UV energy from one instance of a first array of disinfecting UV energy directors, with each disinfecting director of the first array thereof configured to receive disinfecting UV energy from one instance of a first array of disinfecting UV energy emitters; and
a second array of outputs to deliver directed disinfecting UV energy to the at least one second target location, with each output of the second array thereof configured to receive directed disinfecting UV energy from one instance of a second array of disinfecting UV energy directors, with each disinfecting director of the second array thereof configured to receive disinfecting UV energy from one instance of a second array of disinfecting UV energy emitters.

2. The disinfection device as defined in any of the preceding or following clauses, wherein each instance of the disinfecting UV energy directors of the first array thereof, and the corresponding instance of the disinfecting UV energy emitters of the first array thereof are positioned adjacent a common location on a first mounting surface in the housing structure.

3. The disinfection device as defined in any of the preceding or following clauses, wherein each instance of the disinfecting UV energy directors of the second array thereof, and the corresponding instance of the disinfecting UV energy emitters of the second array thereof are positioned adjacent a common location on a second mounting surface in the housing structure.

4. The disinfection device as defined in any of the preceding or following clauses, wherein the outer boundary of the linear housing structure is defined by an outer boundary wall, with each incidence of the first array of outputs and each incidence of the second array of outputs including a passage through the boundary wall.

5. The disinfection device as defined in any of the preceding or following clauses, wherein the first and second arrays of outputs extend along respective axes, with the axes being substantially parallel or substantially in common.

6. The disinfection device as defined in any of the preceding or following clauses, wherein the outer boundary wall has a continuous or discontinuous cross section.

7. The disinfection device as defined in any of the preceding or following clauses, wherein the outer boundary wall has a curved, linear and/or curvilinear cross section.

8. The disinfection device as defined in any of the preceding or following clauses, wherein the boundary layer has at least one ellipsoidal end region, with a pair of pair of opposed wall sections extending therefrom.

9. The disinfection device as defined in any of the preceding or following clauses, wherein the opposed wall sections are oriented with an acute included angle.

10. The disinfection device as defined in any of the preceding or following clauses, wherein the support structure includes a base with an inclined support surface to engage one of the opposed wall sections.

11. The disinfection device as defined in any of the preceding or following clauses, wherein the support structure further comprises a first power supply segment, and the linear housing structure and support structure are each configured with a first set of contacts for coupling the first power supply segment to a second power supply segment in the linear housing structure to energize the first and second arrays of UV energy emitters.

12. The disinfection device as defined in any of the preceding or following clauses, wherein the first and second arrays of UV energy emitters include UV LEDs, and the second power supply segment includes at least one driver to drive the UV LEDs.

13. The disinfection device as defined in any of the preceding or following clauses, wherein the at least one driver is configured to exchange control data signals with a remote controller networked therewith.

14. The disinfection device as defined in any of the preceding or following clauses, wherein the support structure further comprises a first control data path segment, and the linear housing structure and support structure are each configured with a second set of contacts for coupling the first control data path segment to a second control data path segment in the linear housing structure in communication with the at least one driver.

15. A disinfection device for controllably emitting and directing disinfecting UV energy to at least one first target location and at least one second target location in a user space, comprising a linear housing structure mountable on or in at least one support structure and having an outer boundary, wherein the outer boundary is configured to provide:
at least one output configured:
   to receive a first directed disinfecting UV energy from at least one instance of a first array of disinfecting UV energy directors, with each UV energy disinfecting director of the first array thereof configured to receive a first disinfecting UV energy from one instance of a first array of disinfecting UV energy emitters, and to deliver the first directed disinfecting UV energy to the at least one first target location; and
   to receive a second directed disinfecting UV energy from at least one instance of a second array of disinfecting UV energy directors, with each disinfecting UV energy director of the second array thereof configured to receive a second disinfecting UV energy from one instance of a second array of disinfecting UV energy emitters, and to deliver the second directed disinfecting UV energy to the at least one second target location.

16. The disinfection device as defined in any of the preceding or following clauses, wherein each instance of the disinfecting UV energy directors of the first array thereof, and the corresponding instance of the disinfecting UV energy emitters of the first array thereof are positioned adjacent a common location on a first linear mounting surface in the linear housing structure.

17. The disinfection device as defined in any of the preceding or following clauses, wherein each instance of the disinfecting UV energy directors of the second array thereof, and the corresponding instance of the disinfecting UV energy emitters of the second array thereof are positioned adjacent a common location on a second linear mounting surface in the linear housing structure.

18. The disinfection device as defined in any of the preceding or following clauses, wherein the first and second linear surfaces are defined by corresponding first and second linear LED board structures positioned in the linear housing structure.

19. The disinfection device as defined in any of the preceding or following clauses, wherein the outer boundary of the linear housing structure is defined by an outer boundary wall, with each incidence of the array of at least one output including a passage through the boundary wall.

20. The disinfection device as defined in any of the preceding or following clauses, wherein the outer boundary wall has at least one ellipsoidal end region, with a pair of pair of opposed wall sections extending therefrom.

21. The disinfection device as defined in any of the preceding or following clauses, wherein the at least one passage extends through the at least one ellipsoidal end region along a dimension parallel to a central axis thereof, and a dimension transverse to the central axis.

22. The disinfection device as defined in any of the preceding or following clauses, wherein the at least one passage is defined by a pair of opposed linear boundaries which are spaced by included radial angle therebetween.

23. The disinfection device as defined in any of the preceding or following clauses, wherein the first and second linear LED board structures are each configured to extend along a corresponding linear boundary, so as to cause to the first disinfecting UV energy of the first array to pass through the second directed disinfecting UV energy of the second array.

24. The disinfection device as defined in any of the preceding or following clauses, further comprising at least one UV energy transmissive cover structure to close the at least one passage.

25. The disinfection device as defined in any of the preceding or following clauses, wherein the opposed linear boundaries are oriented with an acute included angle relative to the central axis.

26. The disinfection device as defined in any of the preceding or following clauses, wherein the support structure includes a base structure with a support surface to engage one of the opposed wall sections.

27. The disinfection device as defined in any of the preceding or following clauses, wherein the support structure includes a base with a pair of opposed support surfaces, each to engage a corresponding one of the opposed wall sections, so as to position the linear housing structure into the support structure in a wedge-like configuration.

28. The disinfection device as defined in any of the preceding or following clauses, further comprising at least one anchor structure to anchor the linear housing structure in the support structure in the wedge-like configuration.

29. The disinfection device as defined in any of the preceding or following clauses, wherein the linear housing structure and the support structure further comprise complementary communication pathways for transferring power and/or communication signals therebetween.

30. The disinfection device as defined in any of the preceding or following clauses, wherein the support structure and the linear housing structure include respective adjacent surfaces in the wedge-like configuration with the communication pathways defined thereon.

31. A disinfection device for controllably emitting and directing disinfecting UV energy to at least one first proximal target location defined in a first plane, and at least one second distal target location defined in a second plane, in a user space, comprising a housing structure mountable on or in at least one support structure and having an outer boundary, wherein the outer boundary is configured to provide:
at least one output configured:
   to receive a first directed disinfecting UV energy from a first array of at least one first disinfecting UV engine to deliver the first directed disinfecting UV energy to the at least one first proximal target location; and
   to receive a second directed disinfecting UV energy from a second array of second disinfecting UV engines to deliver the second directed disinfecting UV energy to the at least one second proximal target location.

32. The disinfection device as defined in any of the preceding or following clauses, wherein the first array includes at least one instance of the first disinfecting UV engines, and the second array includes at least two instances of the second disinfecting UV engines.

33. The disinfection device of clause 31, wherein instances of first disinfecting UV engines in the first array are fewer in number than instances of second disinfecting UV engines in the second array, so as to accommodate for a relatively shorter distance for the first directed disinfecting UV energy to travel to the first proximal target location, and the relatively longer distance for the second directed disinfecting UV energy to travel to the second distal target location.

34. The disinfection device as defined in any of the preceding or following clauses, wherein the first array of UV engines are spaced at a first spacing, and the second array of UV engines are spaced at a second spacing, wherein the first spacing is greater than the second spacing.

35. The disinfection device as defined in any of the preceding or following clauses, wherein the second spacing is about twice the first spacing.

36. The disinfection device as defined in any of the preceding or following clauses, the first array and second array are aligned with spaced substantially parallel first and second axes.

37. The disinfection device as defined in any of the preceding or following clauses, wherein the housing structure has opposed outer panel structures configured to engage corresponding opposed inner panels structures in a wedge-like heat transfer configuration.

38. The disinfection device as defined in any of the preceding or following clauses, further comprising at least one anchor structure to anchor the linear housing structure in the support structure so as to establish a heat transfer interface between each outer panel structure and a corresponding inner panel structure.

39. The disinfection device as defined in any of the preceding or following clauses, wherein the outer panel structures are in a wedge-like configuration with the opposed inner panel structures.

40. The disinfection device as defined in any of the preceding or following clauses, wherein the housing structure and the support structure further comprise complementary communication pathways for transferring power and/or communication signals therebetween.

41. The disinfection device as defined in any of the preceding or following clauses, wherein the housing structure comprises data storage structure to receive and store authentication data identifying one or more parameters of the linear housing structure.

42. The disinfection device as defined in any of the preceding or following clauses, wherein the housing structure comprises a monitor to update the one or more parameters.

43. The disinfection device as defined in any of the preceding or following clauses, wherein the support structure comprises an authenticator to communicate with a power controller, wherein the authenticator is configured to direct the power controller to deliver power to the first and second arrays when at least one parameter meets a first designated condition, or to interrupt power when at least one parameter meets a second designated condition.

44. The disinfection device as defined in any of the preceding or following clauses, wherein one or more of the first and second disinfecting UV engines includes a UV energy director which receives UV energy from a UV energy emitter.

45. The disinfection device as defined in any of the preceding or following clauses, wherein the UV energy director includes an optical coupling between a UV energy diffuser structure, a UV energy collimator structure, and the UV energy emitter.

46. The disinfection device as defined in any of the preceding or following clauses, wherein one or more of the first target location, the second target location, the housing structure, the support structure, the first mounting surface, the second mounting surface, the first array, the second array, the support structure, the base structure, the opposed support surfaces, the opposed wall sections, the outer panel structures, the inner panel structures is, or are correspondingly, linear in configuration.

47. A disinfection device for controllably emitting and directing disinfecting UV energy to at least one first target region and at least one second target adjacent location in a user space, comprising a housing structure mountable on or in at least one support structure, and having an outer boundary, wherein the outer boundary is configured to provide:

at least one output configured:

to receive a first directed disinfecting UV energy from at least one instance of a first array of disinfecting UV energy directors, with each UV energy disinfecting director of the first array thereof configured to receive a first disinfecting UV energy from one instance of a first array of disinfecting UV energy emitters, and to deliver the first directed disinfecting UV energy to the at least one first target location; and

to receive a second directed disinfecting UV energy from at least one instance of a second array of disinfecting UV energy directors, with each disinfecting UV energy director of the second array thereof configured to receive a second disinfecting UV energy from one instance of a second array of disinfecting UV energy emitters, and to deliver the second directed disinfecting UV energy to the at least one second target location.

48. A disinfection device for controllably emitting and directing disinfecting UV energy to at least one first target region and at least one second target region in an interior user space, comprising a housing structure containing a first array of at least one first disinfecting UV engine configured to deliver a first directed disinfecting UV energy to the at least one first target region, and a second array of at least one second disinfecting UV engine configured to deliver a second directed disinfecting UV energy to the at least one second target region, so as to form a disinfecting UV energy transition between the first and second regions, with each of the first and second disinfecting UV energies at a designated energy value to correspond to corresponding attribute of the first and second regions respectively.

49. A disinfection device for controllably emitting and/or directing disinfecting UV energy to a plurality of regions in an interior or exterior user space, comprising a first array of at least one first disinfecting UV engine configured to deliver a first directed disinfecting UV energy, and a second array of at least one second disinfecting UV engine configured to deliver a second directed disinfecting UV energy, wherein a first group of one or more of the first and/or second disinfecting UV engines is configured to deliver the first and/or second disinfecting energies to at least one first target region, and a second group of one or more of the first and/or second disinfecting UV engines is configured to deliver the first and/or second disinfecting energies to at least one second target region.

50. The disinfection device as defined in any of the preceding or following clauses, comprising at least one housing structure to contain the first and second arrays.

51. The disinfection device as defined in any of the preceding or following clauses, wherein the first and second groups are configured to form a disinfecting UV energy transition between the first and second target regions, with each of the first and second disinfecting UV energies in the respective groups at a designated energy strength according to a corresponding attribute of the first and second regions respectively.

52. The disinfection device as defined in any of the preceding or following clauses, wherein each disinfecting UV engine of the first array is configured to deliver the first directed disinfecting UV energy to a first field, and each disinfecting UV engine of the second array is configured to deliver the second directed disinfecting UV energy to a second field, wherein each of the first target region and second target region includes at least one first field and/or at least one second field.

53. The disinfection device as defined in any of the preceding or following clauses, wherein each of the first and second fields is a proximal field and/or a distal field.

54. The disinfection device as defined in any of the preceding or following clauses, wherein the first group is oriented to deliver the first and/or second directed disinfecting UV energies to the first region, as a central inner sector on a target surface in the interior user space.

55. A disinfection device as defined in any of the preceding or following clauses, wherein the second group is oriented to deliver the first and/or second directed disinfecting UV energy to the second region, as a peripheral outer sector on the target surface in the user space.

56. The disinfection device as defined in any of the preceding or following clauses, wherein the target surface includes a sink, counter, and/or wall surface, at least in part.

57. The disinfection device as defined in any of the preceding or following clauses, wherein the target surface is defined on at least one surface of a wall, floor, door, light, article of furniture, curtain and/or a barrier, fixture, toilet, tub, shower, or a wheeled or non-wheeled assistive device, ATM, parking kiosks, or user accessible key and/or access panel, at least in part.

58. The disinfection device as defined in any of the preceding or following clauses, wherein instances of the first disinfecting UV engine in the first array are equal or unequal in number than instances of the second disinfecting UV engine in the second array, so as to enable collective amounts of the first and second directed disinfecting UV energies to be configured by the corresponding numbers of first and second disinfecting UV engines, at least in part.

59. The disinfection device as defined in any of the preceding or following clauses, wherein the first array includes a plurality of first disinfecting UV engines which are spaced at a first spacing, and the second array of includes a plurality of second disinfecting UV engines which are spaced at a second spacing, wherein the first spacing is greater than, equal to, or less than the second spacing, and wherein each of the disinfecting UV engines of the first or second arrays is respectively interspersed between a pair of disinfecting UV engines of the second or first arrays.

60. The disinfection device as defined in any of the preceding or following clauses, wherein the first spacing is about twice the second spacing.

61. The disinfection device as defined in any of the preceding or following clauses, wherein the first array and second array are aligned with spaced substantially parallel first and second axes.

62. A disinfection device for controllably emitting and directing focused disinfecting UV energy to a plurality of regions in an interior or exterior user space, comprising one or more collections of disinfecting UV engines, configured in one or more groups of one or more disinfecting UV engines to deliver focused respective disinfecting UV energies to at least one first target region and at least one second target region, so as to define:
at least one transition between the first and second target regions, and/or
at least one transition between one of the first and second target regions and an adjacent peripheral region beyond the first and or second regions wherein the adjacent peripheral region receives substantially no disinfecting UV energy.

63. The disinfection device as defined in any of the preceding or following clauses, wherein the transition is defined by a change in disinfecting UV energy strength of a designated value, according to one or more predetermined, algorithmic, formulaic or probable ranges across the transition.

64. The disinfection device as defined in any of the preceding or following clauses, wherein at least one of the first target region, the second target region, the first field, and/or the second field is restricted, least in part.

65. An installation in a user space, comprising the disinfection device as defined in any of the above or below clauses.

66. A method for controllably directing focused disinfecting UV energy to a plurality of regions on a target surface in an interior or exterior user space, comprising:
designating, on the target surface, a plurality of regions;
providing one or more collections of disinfecting UV engines,
configuring the one or more collections in one or more groups of one or more disinfecting UV engines to deliver the corresponding focused disinfecting UV energies to at least one first target region and at least one second target region, so as to define:
at least one transition between the first and second target regions, and/or
at least one transition between one of the first and second target regions and an adjacent peripheral region beyond the first and or second regions wherein the adjacent peripheral region receives substantially no disinfecting UV energy.

67. The method as defined in any of the preceding or following clauses, wherein the designating step includes establishing a grid of sites on the target surface so that at least a plurality of sites are located in each of the first and second target regions, and measuring differences in the disinfecting UV energy according to predetermined, algorithmic, formulaic or probable ranges between them, to define the at least one transition.

68. A linear disinfection device for controllably emitting and directing disinfecting UV energy to at least one first proximal target location defined in a first plane, and at least one second distal target location defined in a second plane, in a user space, comprising a linear housing structure mountable on or in at least one support structure and having an outer boundary, the linear housing structure containing a first array of first disinfecting UV engines configured to deliver a first directed disinfecting UV energy to the at least one first proximal target location, and a second array of second disinfecting UV engines configured to deliver a second directed disinfecting UV energy to the at least one second proximal target location, wherein the outer boundary is configured to provide two or more common outputs extending along the linear housing structure, each of the two or more common outputs configured to receive both the first directed disinfecting UV energy from the first array of first disinfecting UV engines and receive the second directed disinfecting UV energy from the second array of second disinfecting UV engines to deliver the first and second directed disinfecting UV energies to the first and second target 1 locations.

69. The disinfection device as defined in any of the preceding or following clauses, wherein each of the first and second disinfecting UV engines includes a corresponding first and second UV energy director, each of which receives UV energy from a corresponding first and second UV energy emitter.

70. The disinfection device as defined in any of the preceding or following clauses, wherein each of the first and second UV energy directors includes an optical coupling between a corresponding first and second UV energy diffuser structure, a corresponding first and second UV energy collimator structure, and the corresponding first and second UV energy emitter.

71. The disinfection device as defined in any of the preceding or following clauses, wherein instances of the first disinfecting UV engines in the first array are fewer in number than instances of the second disinfecting UV engines in the second array, so as to enable collective amounts of the first and second directed disinfecting UV energy to be configured by the corresponding numbers of first and second disinfecting UV engines.

72. The disinfection device as defined in any of the preceding or following clauses, wherein the first disinfecting UV engines of the first array are spaced at a first spacing, and the second disinfecting UV engines of the second array are spaced at a second spacing, wherein the first spacing is greater than the second spacing, and wherein each of the first disinfecting UV engines is interspersed between a pair of second disinfecting UV engines.

73. The disinfection device as defined in any of the preceding or following clauses, wherein the first spacing is about twice the second spacing.

74. The disinfection device as defined in any of the preceding or following clauses, wherein the first array and second array are aligned with spaced substantially parallel first and second axes.

75. The disinfection device as defined in any of the preceding or following clauses, wherein the linear housing structure and the support structure further comprise complementary communication pathways for transferring power and/or communication signals therebetween.

76. The disinfection device as defined in any of the preceding or following clauses, wherein the linear housing structure comprises data storage structure to receive and store authentication data identifying one or more parameters of the linear housing structure.

77. The disinfection device as defined in any of the preceding or following clauses, wherein the linear housing structure comprises a monitor to update the one or more parameters.

78. The disinfection device as defined in any of the preceding or following clauses, wherein the support structure comprises an authenticator to communicate with a power controller, wherein the authenticator is configured to direct the power controller to deliver power to the first and second arrays when at least one parameter meets a first designated condition, or to interrupt power when at least one parameter meets a second designated condition.

79. The disinfection device as defined in any of the preceding or following clauses, wherein the outer boundary includes an ellipsoidal end region extending along the linear housing structure, with the two or more common outputs in side-by-side relation therein, with opposed outer panel structures extending therefrom.

80. The disinfection device as defined in any of the preceding or following clauses, wherein the opposed outer panel structures are oriented at an acute included angle therebetween.

81. The disinfection device as defined in any of the preceding or following clauses, wherein the outer boundary of the linear housing structure is defined by an outer boundary wall, with the two or more common outputs defined by a corresponding two or more passages through the boundary wall.

82. The disinfection device as defined in any of the preceding or following clauses, wherein the outer boundary wall has at least one ellipsoidal end region, with a pair of opposed outer panel structures extending therefrom.

83. The disinfection device as defined in any of the preceding or following clauses, wherein each of the two or more passages extend through the at least one ellipsoidal end region along a dimension parallel to a central axis thereof, and a dimension transverse to the central axis.

84. The disinfection device as defined in any of the preceding or following clauses, wherein each of the two or more passages is defined by a pair of opposed linear boundaries which are spaced by an acute included angle therebetween.

85. The disinfection device as defined in any of the preceding or following clauses, wherein the first and second disinfecting UV engines include corresponding first and second linear LED board structures, each configured to extend along a corresponding linear boundary, so as to cause to the first disinfecting UV energy of the first array to pass through the second directed disinfecting UV energy of the second array.

86. The disinfection device as defined in any of the preceding or following clauses, further comprising at least one UV energy transmissive cover structure to close each corresponding passage.

87. The disinfection device as defined in any of the preceding or following clauses, wherein the opposed linear boundaries are oriented with an acute included angle relative to the central axis.

88. A disinfection device for controllably emitting and/or directing disinfecting UV energy to a plurality of regions in an interior or exterior user space, comprising a first array of at least one first disinfecting UV engine means configured to deliver a first directed disinfecting UV energy, and a second array of at least one second disinfecting UV engine means configured to deliver a second directed disinfecting UV energy, wherein a first group of one or more of the first and/or second disinfecting UV engine means is configured to deliver the first and/or second disinfecting energies to at least one first target region, and a second group of one or more of the first and/or second disinfecting UV engine means is configured to deliver the first and/or second disinfecting energies to at least one second target region.

89. The disinfection device as defined in any of the preceding or following clauses, comprising at least one housing means to contain the first and second arrays.

90. The disinfection device as defined in any of the preceding or following clauses, wherein each disinfecting UV engine means of the first array is configured to deliver the first directed disinfecting UV energy to a first field, and each disinfecting UV engine means of the second array is configured to deliver the second directed disinfecting UV energy to a second field, wherein each of the first target region and second target region includes at least one first field and/or at least one second field.

91. The disinfection device as defined in any of the preceding or following clauses, wherein the first group is oriented to deliver the first and/or second directed disinfecting UV energies to the first region, as a central inner sector on a target surface in the user space.

92. A disinfection device as defined in any of the preceding or following clauses, wherein the second group is oriented to deliver the first and/or second directed disinfecting UV energy to the second region, as a peripheral outer sector on the target surface in the user space.

93. The disinfection device as defined in any of the preceding or following clauses, wherein instances of the first disinfecting UV engine means in the first array are equal or unequal in number than instances of the second disinfecting UV engine means in the second array, so as to enable collective amounts of the first and second directed disinfecting UV energies to be configured by the corresponding numbers of first and second disinfecting UV engine means, at least in part.

94. The disinfection device as defined in any of the preceding or following clauses, wherein the first array includes a plurality of first disinfecting UV engine means which are spaced at a first spacing, and the second array of includes a plurality of second disinfecting UV engine means of the second array which are spaced at a second spacing, wherein the first spacing is greater than, equal to, or less than the second spacing, and wherein each of the disinfecting UV engine means of the first or second arrays is respectively interspersed between a pair of disinfecting UV engine means of the second or first arrays.

95. A linear disinfection device for controllably emitting and directing disinfecting UV energy to at least one first proximal target location defined in a first plane, and at least one second distal target location defined in a second plane, in a user space, comprising a linear housing means mountable on or in at least one support means and having an outer boundary means, the linear housing means containing a first array of first disinfecting UV engine means configured to deliver a first directed disinfecting UV energy to the at least one first proximal target location, and a second array of second disinfecting UV engine means configured to deliver a second directed disinfecting UV energy to the at least one second proximal target location, wherein the outer boundary means is configured to provide two or more common output means extending along the linear housing means, each of the two or more common output means configured to receive both the first directed disinfecting UV energy from the first array of first disinfecting UV engine means and receive the second directed disinfecting UV energy from the second array of second disinfecting UV engine means to deliver the first and second directed disinfecting UV energies to the first and second target locations.

96. The disinfection device as defined in any of the preceding or following clauses, wherein each of the first and second disinfecting UV engine means includes a corresponding first and second UV energy director means, each of which receives UV energy from a corresponding first and second UV energy emitter means.

97. The disinfection device as defined in any of the preceding or following clauses, wherein each of the first and second UV energy director means includes an optical coupling means between a corresponding first and second UV energy diffuser means, a corresponding first and second UV energy collimator means, and the corresponding first and second UV energy emitter means.

98. The disinfection device as defined in any of the preceding or following clauses, wherein instances of the first disinfecting UV engine means in the first array are fewer in number than instances of the second disinfecting UV engine means in the second array, so as to enable collective amounts of the first and second directed disinfecting UV energy to be configured by the corresponding numbers of first and second disinfecting UV engine means.

99. The disinfection device as defined in any of the preceding clauses, wherein the first disinfecting UV engine means of the first array are spaced at a first spacing, and the second disinfecting UV engine means of the second array are spaced at a second spacing, wherein the first spacing is greater than the second spacing, and wherein each of the first disinfecting UV engine means is interspersed between a pair of second disinfecting UV engine means.

While the present disclosure describes various exemplary embodiments, the disclosure is not so limited. To the contrary, the disclosure is intended to cover various modifications and equivalent arrangements, as will be readily appreciated by the person of ordinary skill in the art.

1. A disinfection device for controllably emitting and directing disinfecting UV energy to at least one elongate first target location and at least one second target location in a user space, comprising a housing structure mountable on a support structure and having an outer boundary, wherein the outer boundary is configured to provide:
a first array of outputs to deliver directed disinfecting UV energy to the at least one first target location, with each output of the first array thereof configured to receive directed disinfecting UV energy from one instance of a first array of disinfecting UV energy directors, with each disinfecting director of the first array thereof configured to receive disinfecting UV energy from one instance of a first array of disinfecting UV energy emitters; and
a second array of outputs to deliver directed disinfecting UV energy to the at least one second target location, with each output of the second array thereof configured to receive directed disinfecting UV energy from one instance of a second array of disinfecting UV energy directors, with each disinfecting director of the second array thereof configured to receive disinfecting UV energy from one instance of a second array of disinfecting UV energy emitters.

2. The disinfection device as defined in claim 1, wherein each instance of the disinfecting UV energy directors of the first array thereof, and the corresponding instance of the disinfecting UV energy emitters of the first array thereof are positioned adjacent a common location on a first mounting surface in the housing structure.

3. The disinfection device as defined in claim 2, wherein each instance of the disinfecting UV energy directors of the second array thereof, and the corresponding instance of the disinfecting UV energy emitters of the second array thereof are positioned adjacent a common location on a second mounting surface in the housing structure.

4. The disinfection device as defined in claim 1, wherein the outer boundary of the linear housing structure is defined by an outer boundary wall, with each incidence of the first array of outputs and each incidence of the second array of outputs including a passage through the boundary wall.

5. The disinfection device as defined in claim 4, wherein the first and second arrays of outputs extend along respective axes, with the axes being substantially parallel or substantially in common.

6. The disinfection device as defined in claim 1, wherein the outer boundary wall has a continuous or discontinuous cross section.

7. The disinfection device as defined in claim 6, wherein the outer boundary wall has a curved, linear and/or curvilinear cross section.

8. The disinfection device as defined in claim 6, wherein the boundary layer has at least one ellipsoidal end region, with a pair of pair of opposed wall sections extending therefrom.

9. The disinfection device as defined in claim 8, wherein the opposed wall sections are oriented with an acute included angle.

10. The disinfection device as defined in claim 9, wherein the support structure includes a base with an inclined support surface to engage one of the opposed wall sections.

11. The disinfection device as defined in claim 1, wherein the support structure further comprises a first power supply segment, and the linear housing structure and support structure are each configured with a first set of contacts for coupling the first power supply segment to a second power supply segment in the linear housing structure to energize the first and second arrays of UV energy emitters.

12. The disinfection device as defined in claim 11, wherein the first and second arrays of UV energy emitters include UV LEDs, and the second power supply segment includes at least one driver to drive the UV LEDs.

13. The disinfection device as defined in claim 12, wherein the at least one driver is configured to exchange control data signals with a remote controller networked therewith.

14. The disinfection device as defined in claim 13, wherein the support structure further comprises a first control data path segment, and the linear housing structure and support structure are each configured with a second set of contacts for coupling the first control data path segment to a second control data path segment in the linear housing structure in communication with the at least one driver.

15. A disinfection device for controllably emitting and directing disinfecting UV energy to at least one first target location and at least one second target location in a user space, comprising a linear housing structure mountable on or in at least one support structure and having an outer boundary, wherein the outer boundary is configured to provide:
at least one output configured:
to receive a first directed disinfecting UV energy from at least one instance of a first array of disinfecting UV energy directors, with each UV energy disinfecting director of the first array thereof configured to receive a first disinfecting UV energy from one instance of a first array of disinfecting UV energy emitters, and to deliver the first directed disinfecting UV energy to the at least one first target location; and
to receive a second directed disinfecting UV energy from at least one instance of a second array of disinfecting UV energy directors, with each disinfecting UV energy director of the second array thereof configured to receive a second disinfecting UV energy from one instance of a second array of disinfecting UV energy emitters, and to deliver the second directed disinfecting UV energy to the at least one second target location.

16. The disinfection device as defined in claim 15, wherein each instance of the disinfecting UV energy directors of the first array thereof, and the corresponding instance of the disinfecting UV energy emitters of the first array thereof are positioned adjacent a common location on a first linear mounting surface in the linear housing structure.

17. The disinfection device as defined in claim 16, wherein each instance of the disinfecting UV energy directors of the second array thereof, and the corresponding instance of the disinfecting UV energy emitters of the second array thereof are positioned adjacent a common location on a second linear mounting surface in the linear housing structure.

18. The disinfection device as defined in claim 17, wherein the first and second linear surfaces are defined by corresponding first and second linear LED board structures positioned in the linear housing structure.

19. The disinfection device as defined in claim 15, wherein the outer boundary of the linear housing structure is defined by an outer boundary wall, with each incidence of the array of at least one output including a passage through the boundary wall.

20. The disinfection device as defined in claim 19, wherein the outer boundary wall has at least one ellipsoidal end region, with a pair of pair of opposed wall sections extending therefrom.

21. The disinfection device as defined in claim 20, wherein the at least one passage extends through the at least one ellipsoidal end region along a dimension parallel to a central axis thereof, and a dimension transverse to the central axis.

22. The disinfection device as defined in claim 21, wherein the at least one passage is defined by a pair of opposed linear boundaries which are spaced by included radial angle therebetween.

23. The disinfection device as defined in claim 22, wherein the first and second linear LED board structures are each configured to extend along a corresponding linear boundary, so as to cause to the first disinfecting UV energy of the first array to pass through the second directed disinfecting UV energy of the second array.

24. The disinfection device as defined in claim 23, further comprising at least one UV energy transmissive cover structure to close the at least one passage.

25. The disinfection device as defined in claim 22, wherein the opposed linear boundaries are oriented with an acute included angle relative to the central axis.

26. The disinfection device as defined in claim 20, wherein the support structure includes a base structure with a support surface to engage one of the opposed wall sections.

27. The disinfection device as defined in claim 20, wherein the support structure includes a base with a pair of opposed support surfaces, each to engage a corresponding one of the opposed wall sections, to position the linear housing structure into the support structure in a wedge-like configuration.

28. The disinfection device as defined in claim 27, further comprising at least one anchor structure to anchor the linear housing structure in the support structure in the wedge-like configuration.

29. The disinfection device as defined in claim 15, wherein the linear housing structure and the support structure further comprise complementary communication pathways for transferring power and/or communication signals therebetween.

30. The disinfection device as defined in claim 29, wherein the support structure and the linear housing structure include respective adjacent surfaces in the wedge-like configuration with the communication pathways defined thereon.

31. A disinfection device for controllably emitting and directing disinfecting UV energy to at least one first proximal target location defined in a first plane, and at least one second distal target location defined in a second plane, in a user space, comprising a housing structure mountable on or in at least one support structure and having an outer boundary, wherein the outer boundary is configured to provide:
at least one output configured:
to receive a first directed disinfecting UV energy from a first array of at least one first disinfecting UV engine to deliver the first directed disinfecting UV energy to the at least one first proximal target location; and
to receive a second directed disinfecting UV energy from a second array of second disinfecting UV engines to deliver the second directed disinfecting UV energy to the at least one second proximal target location.

32. The disinfection device of claim 31, wherein the first array includes at least one instance of the first disinfecting UV engines, and the second array includes at least two instances of the second disinfecting UV engines.

33. The disinfection device of claim 31, wherein instances of first disinfecting UV engines in the first array are fewer in number than instances of second disinfecting UV engines in the second array, so as to accommodate for a relatively shorter distance for the first directed disinfecting UV energy to travel to the first proximal target location, and the relatively longer distance for the second directed disinfecting UV energy to travel to the second distal target location.

34. The disinfection device of claim 33, wherein the first array of UV engines are spaced at a first spacing, and the second array of UV engines are spaced at a second spacing, wherein the first spacing is greater than the second spacing.

35. The disinfection device of claim 34, wherein the second spacing is about twice the first spacing.

36. The disinfection device of claim 31, wherein the first array and second array are aligned with spaced substantially parallel first and second axes.

37. The disinfection device as defined in claim 31, wherein the housing structure has opposed outer panel structures configured to engage corresponding opposed inner panels structures in a wedge-like heat transfer configuration.

38. The disinfection device as defined in claim 37, further comprising at least one anchor structure to anchor the linear housing structure in the support structure so as to establish a heat transfer interface between each outer panel structure and a corresponding inner panel structure.

39. The disinfection device as defined in claim 38, wherein the outer panel structures are in a wedge-like configuration with the opposed inner panel structures.

40. The disinfection device as defined in claim 31, wherein the housing structure and the support structure further comprise complementary communication pathways for transferring power and/or communication signals therebetween.

41. The disinfection device as defined in claim 40, wherein the housing structure comprises data storage structure to receive and store authentication data identifying one or more parameters of the linear housing structure.

42. The disinfection device as defined in claim 41, wherein the housing structure comprises a monitor to update the one or more parameters.

43. The disinfection device of claim 40, wherein the support structure comprises an authenticator to communicate with a power controller, wherein the authenticator is configured to direct the power controller to deliver power to the first and second arrays when at least one parameter meets a first designated condition, or to interrupt power when at least one parameter meets a second designated condition.

44. The disinfection device of claim 31, wherein one or more of the first and second disinfecting UV engines includes a UV energy director which receives UV energy from a UV energy emitter.

45. The disinfection device of claim 44, wherein the UV energy director includes an optical coupling between a UV energy diffuser structure, a UV energy collimator structure, and the UV energy emitter.

46. The disinfection device of any one of claim 35, wherein one or more of the first target location, the second target location, the housing structure, the support structure, the first mounting surface, the second mounting surface, the first array, the second array, the support structure, the base structure, the opposed support surfaces, the opposed wall sections, the outer panel structures, the inner panel structures is, or are correspondingly, linear in configuration.

47. A disinfection device for controllably emitting and directing disinfecting UV energy to at least one first target region and at least one second target adjacent location in a user space, comprising a housing structure mountable on or in at least one support structure, and having an outer boundary, wherein the outer boundary is configured to provide:
at least one output configured:
to receive a first directed disinfecting UV energy from at least one instance of a first array of disinfecting UV energy directors, with each UV energy disinfecting director of the first array thereof configured to receive a first disinfecting UV energy from one instance of a first array of disinfecting UV energy emitters, and to deliver the first directed disinfecting UV energy to the at least one first target location; and
to receive a second directed disinfecting UV energy from at least one instance of a second array of disinfecting UV energy directors, with each disinfecting UV energy director of the second array thereof configured to receive a second disinfecting UV energy from one instance of a second array of disinfecting UV energy emitters, and to deliver the second directed disinfecting UV energy to the at least one second target location.

48. A disinfection device for controllably emitting and directing disinfecting UV energy to at least one first target region and at least one second target region in an interior user space, comprising a housing structure containing a first array of at least one first disinfecting UV engine configured to deliver a first directed disinfecting UV energy to the at least one first target region, and a second array of at least one second disinfecting UV engine configured to deliver a second directed disinfecting UV energy to the at least one second target region, so as to form a disinfecting UV energy transition between the first and second regions, with each of the first and second disinfecting UV energies at a designated energy value to correspond to corresponding attribute of the first and second regions respectively.

49. A disinfection device for controllably emitting and/or directing disinfecting UV energy to a plurality of regions in an interior or exterior user space, comprising a first array of at least one first disinfecting UV engine configured to deliver a first directed disinfecting UV energy, and a second array of at least one second disinfecting UV engine configured to deliver a second directed disinfecting UV energy, wherein a first group of one or more of the first and/or second disinfecting UV engines is configured to deliver the first and/or second disinfecting energies to at least one first target region, and a second group of one or more of the first and/or second disinfecting UV engines is configured to deliver the first and/or second disinfecting energies to at least one second target region.

50. The disinfection device of claim 49, comprising at least one housing structure to contain the first and second arrays.

51. The disinfection device of claim 49, wherein the first and second groups are configured to form a disinfecting UV energy transition between the first and second target regions, with each of the first and second disinfecting UV energies in the respective groups at a designated energy strength according to a corresponding attribute of the first and second regions respectively.

52. The disinfection device of claim 49, wherein each disinfecting UV engine of the first array is configured to deliver the first directed disinfecting UV energy to a first field, and each disinfecting UV engine of the second array is configured to deliver the second directed disinfecting UV energy to a second field, wherein each of the first target region and second target region includes at least one first field and/or at least one second field.

53. The disinfection device of claim 52, wherein each of the first and second fields is a proximal field and/or a distal field.

54. The disinfection device of claim 49, wherein the first group is oriented to deliver the first and/or second directed disinfecting UV energies to the first region, as a central inner sector on a target surface in the interior user space.

55. A disinfection device of claim 54, wherein the second group is oriented to deliver the first and/or second directed disinfecting UV energy to the second region, as a peripheral outer sector on the target surface in the user space.

56. The disinfection device of claim 55, wherein the target surface includes a sink, counter, and/or wall surface, at least in part.

57. The disinfection device of claim 55, wherein the target surface is defined on at least one surface of a wall, floor, door, light, article of furniture, curtain and/or a barrier, fixture, toilet, tub, shower, or a wheeled or non-wheeled assistive device, ATM, parking kiosks, or user accessible key and/or access panel, at least in part.

58. The disinfection device of claim 49, wherein instances of the first disinfecting UV engine in the first array are equal or unequal in number than instances of the second disinfecting UV engine in the second array, so as to enable collective amounts of the first and second directed disinfecting UV energies to be configured by the corresponding numbers of first and second disinfecting UV engines, at least in part.

59. The disinfection device of claim 49, wherein the first array includes a plurality of first disinfecting UV engines which are spaced at a first spacing, and the second array of includes a plurality of second disinfecting UV engines which are spaced at a second spacing, wherein the first spacing is greater than, equal to, or less than the second spacing, and wherein each of the disinfecting UV engines of the first or second arrays is respectively interspersed between a pair of disinfecting UV engines of the second or first arrays.

60. The disinfection device of claim 59, wherein the first spacing is about twice the second spacing.

61. The disinfection device of claim 59, wherein the first array and second array are aligned with spaced substantially parallel first and second axes.

62. A disinfection device for controllably emitting and directing focused disinfecting UV energy to a plurality of regions in an interior or exterior user space, comprising one or more collections of disinfecting UV engines, configured in one or more groups of one or more disinfecting UV engines to deliver focused respective disinfecting UV energies to at least one first target region and at least one second target region, so as to define:
at least one transition between the first and second target regions, and/or
at least one transition between one of the first and second target regions and an adjacent peripheral region beyond the first and or second regions wherein the adjacent peripheral region receives substantially no disinfecting UV energy.

63. The disinfection device of claim 62, wherein the transition is defined by a change in disinfecting UV energy strength of a designated value, according to one or more predetermined, algorithmic, formulaic or probable ranges across the transition.

64. The disinfection device of claim 52, wherein at least one of the first target region, the second target region, the first field, and/or the second field is restricted, least in part.

65. An installation in a user space, comprising the disinfection device of claim 62.

66. A method for controllably directing focused disinfecting UV energy to a plurality of regions on a target surface in an interior or exterior user space, comprising:
designating, on the target surface, a plurality of regions;
providing one or more collections of disinfecting UV engines,
configuring the one or more collections in one or more groups of one or more disinfecting UV engines to deliver the corresponding focused disinfecting UV energies to at least one first target region and at least one second target region, so as to define:
at least one transition between the first and second target regions, and/or
at least one transition between one of the first and second target regions and an adjacent peripheral region beyond the first and or second regions wherein the adjacent peripheral region receives substantially no disinfecting UV energy.

67. The method of claim 66, wherein the designating step includes establishing a grid of sites on the target surface so that at least a plurality of the sites are located in each of the first and second target regions, and measuring differences in the disinfecting UV energy according to predetermined, algorithmic, formulaic or probable ranges between them, to define the at least one transition.

68. A linear disinfection device for controllably emitting and directing disinfecting UV energy to at least one first proximal target location defined in a first plane, and at least one second distal target location defined in a second plane, in a user space, comprising a linear housing structure mountable on or in at least one support structure and having an outer boundary, the linear housing structure containing a first array of first disinfecting UV engines configured to deliver a first directed disinfecting UV energy to the at least one first proximal target location, and a second array of second disinfecting UV engines configured to deliver a second directed disinfecting UV energy to the at least one second proximal target location, wherein the outer boundary is configured to provide two or more common outputs extending along the linear housing structure, each of the two or more common outputs configured to receive both the first directed disinfecting UV energy from the first array of first disinfecting UV engines and receive the second directed disinfecting UV energy from the second array of second disinfecting UV engines to deliver the first and second directed disinfecting UV energies to the first and second target 1 locations.

69. The disinfection device of claim 68, wherein each of the first and second disinfecting UV engines includes a corresponding first and second UV energy director, each of which receives UV energy from a corresponding first and second UV energy emitter.

70. The disinfection device of claim 69, wherein each of the first and second UV energy directors includes an optical coupling between a corresponding first and second UV energy diffuser structure, a corresponding first and second UV energy collimator structure, and the corresponding first and second UV energy emitter.

71. The disinfection device of claim 70, wherein instances of the first disinfecting UV engines in the first array are fewer in number than instances of the second disinfecting UV engines in the second array, so as to enable collective amounts of the first and second directed disinfecting UV energy to be configured by the corresponding numbers of first and second disinfecting UV engines.

72. The disinfection device of claim 71, wherein the first disinfecting UV engines of the first array are spaced at a first spacing, and the second disinfecting UV engines of the second array are spaced at a second spacing, wherein the first spacing is greater than the second spacing, and wherein each of the first disinfecting UV engines is interspersed between a pair of second disinfecting UV engines.

73. The disinfection device of claim 72, wherein the first spacing is about twice the second spacing.

74. The disinfection device of claim 68, wherein the first array and second array are aligned with spaced substantially parallel first and second axes.

75. The disinfection device of claim 68, wherein the linear housing structure and the support structure further comprise complementary communication pathways for transferring power and/or communication signals therebetween.

76. The disinfection device of claim 75, wherein the linear housing structure comprises data storage structure to receive and store authentication data identifying one or more parameters of the linear housing structure.

77. The disinfection device of claim 76, wherein the linear housing structure comprises a monitor to update the one or more parameters.

78. The disinfection device of claim 77, wherein the support structure comprises an authenticator to communicate with a power controller, wherein the authenticator is configured to direct the power controller to deliver power to the first and second arrays when at least one parameter meets a first designated condition, or to interrupt power when at least one parameter meets a second designated condition.

79. The disinfection device of claim 68, wherein the outer boundary includes an ellipsoidal end region extending along the linear housing structure, with the two or more common outputs in side-by-side relation therein, with opposed outer panel structures extending therefrom.

80. The disinfection device of claim 79, wherein the opposed outer panel structures are oriented at an acute included angle therebetween.

81. The disinfection device of claim 68, wherein the outer boundary of the linear housing structure is defined by an outer boundary wall, with the two or more common outputs defined by a corresponding two or more passages through the boundary wall.

82. The disinfection device of claim 81, wherein the outer boundary wall has at least one ellipsoidal end region, with a pair of opposed outer panel structures extending therefrom.

83. The disinfection device of claim 82, wherein each of the two or more passages extend through the at least one ellipsoidal end region along a dimension parallel to a central axis thereof, and a dimension transverse to the central axis.

84. The disinfection device of claim 83, wherein each of the two or more passages is defined by a pair of opposed linear boundaries which are spaced by an acute included angle therebetween.

85. The disinfection device of claim 72, wherein the first and second disinfecting UV engines include corresponding first and second linear LED board structures, each configured to extend along a corresponding linear boundary, so as to cause to the first disinfecting UV energy of the first array to pass through the second directed disinfecting UV energy of the second array.

86. The disinfection device of claim 85, further comprising at least one UV energy transmissive cover structure to close each corresponding passage.

87. The disinfection device of claim 86, wherein the opposed linear boundaries are oriented with an acute included angle relative to the central axis.
